(19) United States
(12) Patent Application Publication
(10) Pub. No.: US 2021/0052701 A1
(43) Pub. Date: Feb. 25, 2021

(54) Methods of Treating Vascular Leakage Using CXCL12 Peptides

(71) Applicants: The Medical College of Wisconsin, Inc., Milwaukee, WI (US); Loyola University Chicago, Maywood, IL (US)

(72) Inventors: Brian F. Volkman, Milwaukee, WI (US); Matthias Majetschak, Maywood, IL (US); You-Hong Cheng, Maywood, IL (US)

(21) Appl. No.: 17/081,180

(22) Filed: Oct. 27, 2020

Related U.S. Application Data
(63) Continuation of application No. 16/434,701, filed on Jun. 7, 2019, now abandoned.
(60) Provisional application No. 62/682,442, filed on Jun. 8, 2018.

(51) Int. Cl. A61K 38/19; A61P 11/00; A61P 9/14

(52) U.S. Cl. CPC A61K 38/195; A61P 9/14; A61P 11/00

(57) ABSTRACT

The present invention relates to methods for treatment of capillary leak syndrome and acute respiratory distress syndrome using CXCL12 peptides, specifically a constitutively monomeric CXCL12 peptide or a CXCL12 locked dimer polypeptide.

### CROSS-REFERENCE TO RELATED APPLICATIONS

This application is a continuation of U.S. application Ser. No. 16/434,701, filed Jun. 7, 2019, which claims priority to U.S. Provisional Application No. 62/682,442 filed on Jun. 8, 2018, the contents of which are incorporated by reference in its entirety.

### STATEMENT REGARDING FEDERALLY SPONSORED RESEARCH

This invention was made with government support under grants R01 AI058072 and GM107495-01A1, awarded by the National Institutes of Health and W81XWH-15-1-0262 awarded by the Department of Defense USAMRMC/USAMRAA. The government has certain rights in the invention.

### BACKGROUND

Acute respiratory distress syndrome (ARDS) remains a major contributor to morbidity and mortality in critically ill patients. It is generally accepted that mild ARDS and its progression into moderate and severe ARDS is caused by local and systemic coagulation and inflammation, which leads to impaired pulmonary endothelial barrier function, third spacing of fluids into the lung and formation of lung edema, the hallmark of ARDS. Thrombin plays an important role in the pathogenesis of ARDS; in addition to functions of thrombin in the clotting cascade, thrombin fulfills diverse roles in inflammation and is well known to impair endothelial barrier function through activation of the G protein-coupled receptors (GPCR) protease-activated receptors (PARs). All four members of the PAR family (PAR1-4) can be activated by thrombin. A large body of evidence suggests that PAR-1 is the major mediator of thrombin signaling in vascular endothelial cells. PAR-1 is activated when thrombin cleaves its extracellular N-terminal domain between residues Arg-41 and Ser-42, which unmasks a new N-terminus that serves as a tethered ligand. Drugs that limit impairment of the lung endothelial barrier by thrombin, however, are not available, but desirable for their potential to improve outcomes.

Recently, administration of cognate, non-cognate and synthetic chemokine (C-X-C) motif receptor (CXCR) 4 agonists has been shown to attenuate lung injury in various experimental models and CXCL12 (stromal cell-derived factor-1a), the cognate agonist of CXCR4 and atypical chemokine receptor 3 (ACKR3), has been described to attenuate thrombin-induced impairment of endothelial cell barrier function. A more detailed pharmacological characterization of these CXCL12-mediated effects, however, is lacking and the effects of other CXCR4/ACKR3 ligands on lung endothelial cell barrier function are ill defined. Moreover, information on the structural requirements of CXCL12 to attenuate thrombin-mediated lung endothelial barrier disruption and the relationship to its CXCR4/ACKR3 agonist activity is not available.

### SUMMARY OF THE INVENTION

In one aspect, the present invention provides a method of treating capillary leakage syndrome in a subject in need thereof comprising administering to the subject a therapeutically effective amount of a composition comprising a constitutively monomeric CXCL12 peptide comprising the amino acid sequence of SEQ ID NO:1 wherein the amino acids at positions 55 and 58 are substituted with cysteine to treat capillary leakage syndrome.

In another aspect, the present invention provides a method of treating acute respiratory distress syndrome (ARDS) in a subject in need thereof comprising administering to the subject a therapeutically effective amount of a composition comprising a constitutively monomeric CXCL12 peptide comprising the amino acid sequence of SEQ ID NO:1 wherein the amino acids at positions 55 and 58 are substituted with cysteine to treat the ARDS.

In another aspect, the disclosure provides a method of treating capillary leakage syndrome in a subject in need thereof comprising administering to the subject a therapeutically effective amount of a composition comprising a CXCL12a locked dimer polypeptide comprising two monomers locked together by covalent bond to treat capillary leakage syndrome.

In yet another aspect, the disclosure provides a method of treating acute respiratory distress syndrome (ARDS) in a subject in need thereof comprising administering to the subject a therapeutically effective amount of a composition comprising a CXCL12a locked dimer polypeptide comprising two monomers locked together by covalent bond to treat the ARDS.

### BRIEF DESCRIPTION OF DRAWINGS

The patent or patent application file contains at least one drawing in color. Copies of this patent or patent application publication with color drawings will be provided by the Office upon request and payment of the necessary fee.

FIGS. 1A-1B show expression of CXCR4, ACKR3 and CXCR4:ACKR3 heteromers on hPPAEC and effects of CXCR4/ACKR3 ligands on hPPAEC monolayer permeability. (A) Detection of CXCR4, ACKR3 and CXCR4:ACKR3 heteromers on hPPAEC by PLA. Typical PLA images for the detection of individual receptors and CXCR4:ACKR3 heteromers. Images show merged PLA/40,6-diamidino-2-phenylindole dihydrochloride (DAPI) signals. Ctrl: Omission of one secondary antibody. (B) hPPAEC were grown to a confluent monolayer on collagen-coated permeable membranes. Cells were then exposed to vehicle or 50 nM of CXCR4/ACKR3 ligands for 10 minutes, as indicated, followed by the addition of FITC-dextran. Endothelial permeability was assessed by measuring the amount of FITC-dextran that permeated through the cell monolayer. N=3 in quadruplicate. No cells: 100% permeability, open squares. RFU: Relative fluorescence units. *: p<0.05 vs. vehicle (2-way ANOVA/Bonferroni's multiple comparison post hoc test).

FIGS. 2A-2B show impairment of hPPAEC monolayer permeability by thrombin. (A) hPPAEC were grown to a confluent monolayer on collagen-coated permeable membranes and then exposed to different concentration of thrombin for 10 min, followed by the addition of FITC-dextran. Endothelial permeability was assessed by measuring the amount of FITC-dextran that permeated through the cell monolayer. No cells: 100% permeability. RFU: Relative fluorescence units. N=3 in quadruplicate. (B) Dose-response curves for thrombin-induced permeability, data from A. 100% permeability=permeability in the absence of hPPAEC. Open squares: Permeability at t=55 min. Light grey squares: Permeability at t=135 min. Dark grey squares: Permeability at t=255 min. Dose-response curves were generated using nonlinear regression analyses.

FIGS. 3A-3D show effects of CXCL12 and ubiquitin on thrombin-induced impairment of hPPAEC monolayer permeability. hPPAEC were grown to a confluent monolayer on collagen-coated permeable membranes. (A) hPPAEC were pre-treated with vehicle, 100 nM of CXCL12 or ubiquitin for 10 minutes, as indicated, and then exposed to thrombin (50 nM), followed by the addition of FITC-dextran. Vehicle: no thrombin. Endothelial permeability was assessed by measuring the amount of FITC-dextran that permeated through the cell monolayer. No cells: 100% permeability (open circles). RFU: Relative fluorescence units. N=3 in quadruplicate. *: p<0.05 vs. vehicle/thrombin. (B-D) hPPAEC were exposed to 35 nM of thrombin or vehicle. After 10 min, thrombin-exposed cells were treated with vehicle, CXCL12 (50 nM) and/or AMD3100 (10 μM) (B), with vehicle, ubiquitin (50 nM) and/or AMD3100 (10 μM) (C) or with various concentrations of ubiquitin (D) followed by the addition of FITC-dextran. The experimental conditions are indicated. Endothelial permeability was assessed by measuring the amount of FITC-dextran that permeated through the cell monolayer. RFU: Relative fluorescence units. N=3 in quadruplicate. *: p<0.05 vs. thrombin/vehicle (2-way ANOVA/Bonferroni's multiple comparison post hoc test).

FIGS. 4A-4D show effects of CXCL12 and ubiquitin on thrombin-induced impairment of HULEC-5a monolayer permeability. (A) HULEC-5a were grown to a confluent monolayer on collagen-coated permeable membranes and then exposed to different concentration of thrombin for 10 min, followed by the addition of FITC-dextran. Endothelial permeability was assessed by measuring the amount of FITC-dextran that permeated through the cell monolayer. No cells: 100% permeability. RFU: Relative fluorescence units. N=3 in quadruplicate. (B) Dose-response curves for thrombin-induced permeability, data from A. 100% permeability=permeability in the absence of HULEC-5a. Open squares: Permeability at t=55 min. Light grey squares: Permeability at t=135 min. Dark grey squares: Permeability at t=255 min. (C/D) HULEC-5a were grown to a confluent monolayer on collagen-coated permeable membranes and then exposed to 50 nM of thrombin or vehicle. After 10 min, thrombin-exposed cells were treated with vehicle, CXCL12 (50 nM) (C) or ubiquitin (50 nM) (D), followed by the addition of FITC-dextran. The experimental conditions are indicated. Endothelial permeability was assessed by measuring the amount of FITC-dextran that permeated through the cell monolayer. RFU: Relative fluorescence units. N=3 in quadruplicate. *: p<0.05 vs. thrombin/vehicle (2-way ANOVA/Bonferroni's multiple comparison post hoc test).

FIG. 5 shows electrophoretic mobility of CXCL12α, CXCL12₁ and CXCL12₂. Per lane 1 μg of protein in 25 μL sample buffer (4 μM) were used for SDS-polyacrylamide gel electrophoresis under non-reducing (−) and reducing (+, (WE: 0.357 M β-mercaptoethanol) conditions. The position of molecular mass standards is indicated on the left.

FIGS. 6A-6F show Presto-Tango β-arrestin 2 recruitment assays for CXCR4 (A-C) and ACKR3 (D-F). RLU %: % of the luminescence signal for 1 μM CXCL12α. N=9 for CXCL12a and n=3 for all other proteins.

FIGS. 7A-7D show dose-dependent effects of CXCL12α/β, CXCL12 (3-68) and CXCL12 mutants K27A/R41A/R47A, R47E and S-S4V on thrombin-induced impairment of hPPAEC monolayer permeability. hPPAEC cells were grown to a confluent monolayer on collagen-coated permeable membranes. hPPAEC were then exposed to 35 nM of thrombin. After 10 min, thrombin-exposed cells were treated with vehicle or 50 nM (A), 5 nM (B), 0.5 nM (C) or 0.05 nM (D) of the various proteins, as indicated. In (D) 5 nM CXCL12a was used as a positive control. N=3 in quadruplicate.

FIGS. 8A-8D show dose-dependent effects of CXCL12α, CXCL12₁ and CXCL12₂ on thrombin-induced impairment of hPPAEC monolayer permeability. hPPAEC cells were grown to a confluent monolayer on collagen-coated permeable membranes. hPPAEC were then exposed to 35 nM of thrombin. After 10 min, thrombin-exposed cells were treated with vehicle or 50 nM (A), 5 nM (B), 0.5 nM (C) or 0.05 nM (D) of the various proteins, as indicated. In (D) 5 nM CXCL12α was used as a positive control. N=3 in quadruplicate.

FIGS. 9A-9B show inhibition of thrombin-induced hyper-permeability of hPPAEC by CXCL12/CXCL12 variants. % inhibition: % inhibition of thrombin-induced hyperpermeability. Data from FIGS. 7 and 8 at t=255 min. N=3 in quadruplicate. *: p<0.05 vs. CXCL12a (2-way ANOVA/Bonferroni's multiple comparison post hoc test).

FIG. 10 includes Table 1, which shows CXCR4 and ACKR3 activity of CXCL12/CXCL12 variants-PRESTO-Tango.

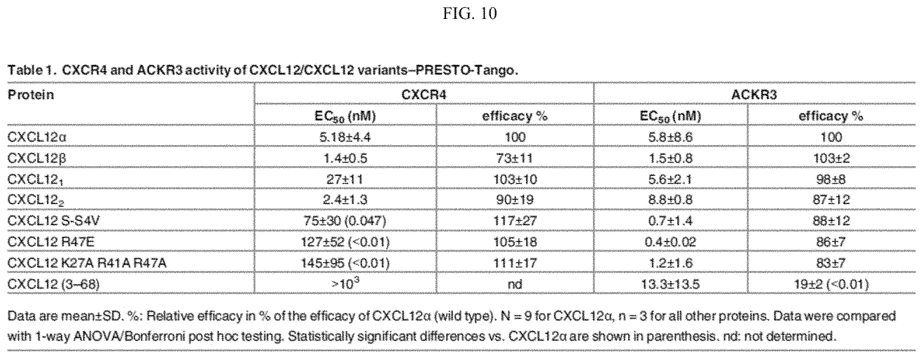

FIG. 11 demonstrates the effects of CXCL12 and engineered CXCR4 agonists on the development of ARDS. Animals underwent 70 min of left lung ischemia for 70 min plus hemorrhage to a MAP of 40 mmHg for the last 30 min of ischemia, followed by fluid resuscitation to a MAP of 60 mmHg until t=300 min. Arrow: drug injection. Dashed lines indicate the P:F ratio (mmHg) threshold values for the diagnosis of mild, moderate and severe ARDS. Vehicle: n=5. CXCL12: n=3. CXCL12: n=4. CXCL12-LM: n=3, CXCL12-LD: n=4. CXCL12 K27A/R41A/R47A: n=3. LI+HEM: lung ischemia reperfusion injury plus hemorrhage. Data are mean±SEM. *: p<0.05 vs. vehicle (2-way ANOVA/Dunnett's multiple comparisons test).

FIGS. 12A-12C show lung histology after treatment with CXCL12 and engineered CXCR4 agonists A. Lung specimens were placed in formalin fixative solution and were then embedded in paraffin wax, sliced into 5 μm sections and stained with H&E. Representative images from H&E stained lung sections from animals after vehicle (left) and CXCL12 (0.7 μmol/kg, right treatment. B/C. Lung injury scores (B. uninjured (left) lung; C. injured (right) lung). Data are median±interquartile ranges. Data were analyzed with the Kruskal Wallis test with Dunn's multiple comparisons test. The level of statistical significance (p) vs. ctrl. (vehicle treatment) indicated.

### DETAILED DESCRIPTION OF THE INVENTION

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The present invention provides a constitutively monomeric CXCL12 variant (CXCL12₁) engineered to resist peptide-induced dimerization by maintaining steric repulsion of the chemokine helix. Six short CXCR4 peptides, centered on Tyr7, Tyr12, or Tyr21, were synthesized to study the contributions of individual sulfotyrosines in peptide binding and specificity. Peptides were titrated into CXCL12_{WT}, CXCL12₂ (a constitutively dimeric variant of CXCL12 containing L36C/A65C mutations), or CXCL12₁ and the interaction was monitored by 2D NMR. While sulfopeptides encompassing sTyr7 and sTyr12 interacted nonspecifically, an unsulfated Tyr7 peptide induced a new set of chemical shift perturbations in the CXCL12 monomer that were also observed upon binding of the intact CXCR4₁₋₃₈ N-terminal domain. In contrast, the Tyr21 peptides bound specifically to the sTyr21 recognition site in all three CXCL12 variants, but exhibited a significantly higher affinity for CXCL12₂. The sTyr21 sulfopeptide correspondingly increased the CXCL12 dimerization affinity by eight-fold, revealing an allosteric coupling between the sulfotyrosine binding site and CXCL12 dimerization.

Tyrosine sulfation is a post-translational modification that enhances protein-protein interactions and may identify druggable sites in the extracellular space. The G protein-coupled receptor CXCR4 is a prototypical example with three potential sulfation sites at positions 7, 12 and 21. Each receptor sulfotyrosine participates in specific contacts with its chemokine ligand in the structure of a soluble, dimeric CXCL12:CXCR4(1-38) complex, but their relative importance for CXCR4 binding and activation by the monomeric chemokine remains undefined. NMR titrations with short sulfopeptides showed that the tyrosine motifs of CXCR4 varied widely in their contributions to CXCL12 binding affinity and site specificity. Whereas the Tyr21 sulfopeptide bound the same site as in previously solved structures, the Tyr7 and Tyr12 sulfopeptides interacted nonspecifically. Surprisingly, the unsulfated Tyr7 peptide occupied a hydrophobic site on the CXCL12 monomer that is inaccessible in the CXCL12 dimer.

Functional analysis of CXCR4 mutants validated the relative importance of individual CXCR4 sulfotyrosine modifications (Tyr21>Tyr12>Tyr7) for CXCL12 binding and receptor activation. Biophysical measurements also revealed a cooperative relationship between sulfopeptide binding at the Tyr21 site and CXCL12 dimerization, the first example of allosteric behavior in a chemokine. Future ligands that occupy the sTyr21 recognition site may act as both competitive inhibitors of receptor binding and allosteric modulators of chemokine function. Together, our data suggests that sulfation does not ubiquitously enhance complex affinity and that distinct patterns of tyrosine sulfation could encode oligomer selectivity—implying another layer of regulation for chemokine signaling.

CXCL12₁ Monomer.

In one embodiment, the invention provides a constitutively monomeric CXCL12 variant, termed CXCL12₁, engineered to resist peptide-induced dimerization by maintaining steric repulsion of the chemokine helix. Specifically, the monomeric CXCL12 peptide (CXCL12₁, SEQ ID NO:2) of the present invention has been modified to exhibit at least L55C and I58C substitutions relative to wild-type CXCL12 (SEQ ID NO:1). Other substitutions are also contemplated by this invention. The CXCL12₁ monomer is described in detailed in U.S. Patent Publication US 2015/0361152 and U.S. Pat. No. 9,908,923, which are incorporated herein by reference in their entireties.

Wild-type CXCL12 (SEQ ID NO:1): KPVSLSYRCPCRFFESHVARANVKHLKILN TPNCALQIVA RLKNNNRQVC IDPKLKWIQE YLEKALNK

CXCL12₁ monomer (SEQ ID NO:2): KPVSLSYRCPCRFFESHVAR ANVKHLKILN TPNCALQIVA RLKNNNRQVC IDPKCKWCQEYLEKALNK

In one embodiment, the CXCL12₁ monomer of the present invention comprises a substantially pure preparation. By “substantially pure” we mean a preparation in which more than 90%, e.g., 95%, 98% or 99% of the preparation is that of the CXCL12₁ monomer.

The CXCL12₁ monomer of the present invention could also be incorporated into a larger protein or attached to a fusion protein that may function to increase the half-life of the monomer in vivo or be used as a mechanism for time released and/or local delivery (U.S. Patent Publication No. 20060088510). In another embodiment, the invention provides an isolated CXCL12₁ monomer as described above. By “isolated” we mean a nucleic acid sequence that is identified and separated from at least one component or contaminant with which it is ordinarily associated. An isolated nucleic acid is present in a form or setting that is different from that in which it is found in nature. In contrast, non-isolated nucleic acids such as DNA and RNA are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, an isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide can be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide can be single-stranded), but can contain both the sense and anti-sense strands (i.e., the oligonucleotide or polynucleotide can be double-stranded).

The CXCL12₁ monomer of the present invention can be prepared by standard techniques known in the art. The peptide component of CXCL12 is composed, at least in part, of a peptide, which can be synthesized using standard techniques such as those described in Bodansky, M. Principles of Peptide Synthesis, Springer Verlag, Berlin (1993) and Grant, G. A. (ed.). Synthetic Peptides: A User's Guide, W. H. Freeman and Company, New York (1992). Automated peptide synthesizers are commercially available (e.g., Advanced ChemTech Model 396; Milligen/Biosearch 9600). Additionally, one or more modulating groups can be attached to the CXCL12 derived peptidic component by standard methods, such as by using methods for reaction through an amino group (e.g., the alpha-amino group at the amino-terminus of a peptide), a carboxyl group (e.g., at the carboxy terminus of a peptide), a hydroxyl group (e.g., on a tyrosine, serine or threonine residue) or other suitable reactive group on an amino acid side chain (see e.g., Greene, T. W. and Wuts, P. G. M. Protective Groups in Organic Synthesis, John Wiley and Sons, Inc., New York (1991)). Exemplary syntheses of preferred CXCL12₁ monomer according to the present invention are described further in the Examples below.

Peptides of the invention may be chemically synthesized using standard techniques such as those described in Bodansky, M. Principles of Peptide Synthesis, Springer Verlag, Berlin (1993) and Grant, G. A. (ed.). Synthetic Peptides: A User's Guide, W. H. Freeman and Company, New York, (1992) (all of which are incorporated herein by reference).

In another aspect of the invention, peptides may be prepared according to standard recombinant DNA techniques using a nucleic acid molecule encoding the peptide. A nucleotide sequence encoding the peptide can be determined using the genetic code and an oligonucleotide molecule having this nucleotide sequence can be synthesized by standard DNA synthesis methods (e.g., using an automated DNA synthesizer). Alternatively, a DNA molecule encoding a peptide compound can be derived from the natural precursor protein gene or cDNA (e.g., using the polymerase chain reaction (PCR) and/or restriction enzyme digestion) according to standard molecular biology techniques.

CXCL12₂ Locked Dimer.

In one embodiment, the invention provides methods of using a CXCL12-α2 locked dimer polypeptide comprising at least two monomers locked together by a covalent bond. By “locked” we mean the monomer components of the polypeptide are linked to each other via at least one covalent bond (e.g., a disulfide bond). The monomer and dimer forms do not interconvert. In a preferred embodiment, at least one of residues L36 and A65 of the wild type CXCL12 monomer sequence (SEQ ID NO:1) is replaced with cysteine residues to create at least one intermolecular disulfide bond between cysteine residues at position 36 of one subunit and/or position 65 of the other monomer subunit. Either or both cysteine residues at positions L36 and A65 can be replaced with cysteines to form the locked dimer with at least one, but preferably two, disulfide bonds.

The monomers of the locked dimer may be identical or may be non-identical. In one embodiment, at least one of the monomers has the amino acid sequence comprising SEQ ID NO:3. In alternate embodiments, both monomers have the amino acid sequence comprising SEQ ID NO:3. The CXCL12α2 locked dimer polypeptide is also described in U.S. Pat. Nos. 9,346,871, 8,524,670, and 7,923,016, each of which is incorporated herein as if set forth in its entirety.

Other residue(s) besides L36 and A65 in CXCL12 (SEQ ID NO:3) could be mutated to cysteines in order to form the locked dimer similar to the one of the present invention. For instance, a locked dimer can be created by mutating amino acid(s) in the CXCL12 dimer interface to cysteines that are positioned opposite one another yielding a disulfide bond that covalently links two CXCL12 monomers. For example, residue K27 is directly across the CXCL12 dimer interface from residue K27 of the opposing subunit and K27C mutation would likely make a locked dimer. Residues L26 and 128 are also on the CXCL12 dimer interface, and a L26C/I28C variant should form a locked dimer with L26C of one monomer subunit forming a disulfide bond with I28C of the opposing subunit and I28C of one monomer subunit forming a disulfide bond with L26C of the opposing subunit. All proposed cysteine mutations are numbered relative to SEQ ID NO:1.

Locked dimer CXCL12₂ (SEQ ID NO:3):

KPVSLSYRCPCRFFESHVARANVKHLKILNTPNCACQIVARLKNNNRQVC IDPKLKWIQE YLEKCLNK (bold C are mutated LL36 and A65 positions)

In a preferred embodiment, the CXCL12-α2 locked dimer of the present invention has substitutions at both L36C/A65C residues relative to SEQ ID NO:1, for examples as shown in SEQ ID NO:3. A similar locked dimer could be created using disulfide bonds introduced between beta strand 1 and the middle of the alpha helix. For example, CXCL12 with I28C/Y61C or I28C/L62C would form a locked dimer with beta strand one of one monomer having a disulfide bond to the middle of the alpha helix of the second monomer thus making a locked dimer. Additionally, a locked dimer may be created by generating a construct that produces two CXCL12 monomers where the C-terminus of one is linked to the N-terminus of the other through an amino acid linker.

Additional methods for making locked dimers of CXCL12 could also include other types of covalent linkages besides disulfide bonds including, but not limited to, chemical cross-linking reagents.

In a preferred embodiment, the locked dimer of the present invention comprises a substantially pure preparation. By “substantially pure” we mean a preparation in which more than 90%, e.g., 95%, 98% or 99% of the preparation is that of the locked dimer.

In a preferred embodiment, at least one of the monomers comprising the locked dimer of the present invention has the amino acid sequence as shown in SEQ ID NO:3 or a homologue or fragment thereof. In a further preferred embodiment, the dimer comprises two monomers having the amino acid sequence as shown in SEQ ID NO:3 or a homologue or variant thereof. By “homologue” we mean an amino acid sequence generally being at least 80%, preferably at least 90% and more preferably at least 95% identical to the polypeptide of SEQ ID NO:3 over a region of at least twenty contiguous amino acids. By “fragment,” we mean peptides, oligopeptides, polypeptides, proteins and enzymes that comprise a stretch of contiguous amino acid residues, and exhibit substantially a similar, but not necessarily identical, functional activity as the complete sequence. Fragments of SEQ ID NO:3, or their homologues, will generally be at least ten, preferably at least fifteen, amino acids in length, and are also encompassed by the term “a CXCL12 monomer” as used herein.

Mutations known to prevent degradation of CXCL12 or to increase the in vivo half-life may also be incorporated into the CXCL12-α2 sequence. For instance, adding a serine to the N-terminus along with a S4V substitution prevents CXCL12 degradation by proteases. Therefore, adding a serine to the N-terminus would likely similarly prevent protease degradation of the CXCL12-α2 locked dimer of the present invention.

The CXCL12-α2 locked dimer also binds heparin. Amino acid substitutions in CXCL12, including K24S, K27S, or K24S/K27S can prevent heparin binding and increase the half-life of CXCL12 in vivo; therefore, similar mutations in CXCL12-α2 would likely prevent heparin binding and increase the in vivo half-life of the dimer.

CXCL12-α2 variants have been generated that have a Gly-Met dipeptide on the N-terminus. N-terminal extensions to CXCL12 prevent CXCR4 activation and their presence in CXCL12-α2 may increase its effectiveness. Additionally, it may be useful to create CXCL12-α2 variants where both subunits are not identical. For example, only one monomer of the CXCL12-α2 dimer may need to include an added N-terminal serine and a S4V substitution or the K24S, K27S, or K24S/K27S substitutions to prevent heparin binding. Alternatively, a CXCL12-α2 variant where the N-terminus of one monomer has the native sequence but the other has been extended may have different or enhanced pharmacological properties compared to CXCL12-α2.

The locked CXCL12 dimer could also be incorporated into a larger protein or attached to a fusion protein that may function to increase the half-life of the dimer in vivo or be used as a mechanism for time released and/or local delivery (U.S. Patent Appl. No. 20060088510).

CXCL12-α2 locked dimer polypeptides can be prepared by standard techniques known in the art. The peptide component of CXCL12-α2 is composed, at least in part, of a peptide, which can be synthesized using standard techniques such as those described in Bodansky, M., Principles of Peptide Synthesis, Springer Verlag, Berlin (1993) and Grant, G. A. (ed.). Synthetic Peptides: A User's Guide, W.H. Freeman and Company, New York (1992). Automated peptide synthesizers are commercially available (e.g., Advanced ChemTech Model 396; Milligen/Biosearch 9600). Additionally, one or more modulating groups can be attached to the CXCL12-α2 derived peptidic component by standard methods, such as by using methods for reaction through an amino group (e.g., the alpha-amino group at the amino-terminus of a peptide), a carboxyl group (e.g., at the carboxy terminus of a peptide), a hydroxyl group (e.g., on a tyrosine, serine or threonine residue) or other suitable reactive group on an amino acid side chain (see e.g., Greene, T. W. and Wuts, P. G. M. Protective Groups in Organic Synthesis, John Wiley and Sons, Inc., New York (1991)), as described in U.S. Pat. No. 9,346,871, the contents of which are incorporated by reference.

Peptides of the invention may be chemically synthesized using standard techniques such as those described in Bodansky, M. Principles of Peptide Synthesis, Springer Verlag, Berlin (1993) and Grant, G. A. (ed.). Synthetic Peptides: A User's Guide, W.H. Freeman and Company, New York, (1992) (all of which are incorporated herein by reference).

In another aspect of the invention, peptides may be prepared according to standard recombinant DNA techniques using a nucleic acid molecule encoding the peptide. A nucleotide sequence encoding the peptide can be determined using the genetic code and an oligonucleotide molecule having this nucleotide sequence can be synthesized by standard DNA synthesis methods (e.g., using an automated DNA synthesizer). Alternatively, a DNA molecule encoding a peptide compound can be derived from the natural precursor protein gene or cDNA (e.g., using the polymerase chain reaction (PCR) and/or restriction enzyme digestion) according to standard molecular biology techniques.

CXCL12₁ and CXCL12₂ Pharmaceutical Compositions.

In another embodiment, the invention provides a composition comprising a substantially pure CXCL12₁ monomer o or CXCL12₂ dimer, and a pharmaceutically acceptable carrier. By “pharmaceutically acceptable carrier” we mean any and all solvents, dispersion media, coatings, antibacterial and antifungal agents, isotonic and absorption delaying agents, and the like that are physiologically compatible. In one embodiment, the carrier may be suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, membrane nanoparticle or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, such as, monostearate salts and gelatin.

Moreover, the CXCL12₁ monomer or CXCL12₂ dimer can be administered in a time-release formulation, such as in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound (e.g. CXCR4 agonist) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. The CXCL12₁ monomer or CXCL12₂ dimer also may be formulated with one or more additional compounds that enhance the solubility of the CXCL12₁ monomer or CXCL12₂ dimer.

Administration.

The CXCL12₁ monomer or CXCL12₂ dimer of the present invention, optionally comprising other pharmaceutically active compounds, can be administered to a patient orally, rectally, parenterally, (e.g., intravenously, intramuscularly, or subcutaneously) intracisternally, intravaginally, intraperitoneally, intravesically, locally (for example, powders, ointments or drops), or as a buccal or nasal spray. Other contemplated formulations include projected nanoparticles, liposomal preparations, resealed erythrocytes containing the active ingredient, and immunologically-based formulations.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound, and the like. In particular, parenteral administration includes subcutaneous, intraperitoneal, intravenous, intraarterial, intramuscular, or intrasternal injection and intravenous, intraarterial, or kidney dialytic infusion techniques.

Compositions suitable for parenteral injection comprise the CXCL12₁ monomer or CXCL12₂ dimer of the invention combined with a pharmaceutically acceptable carrier such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols (e.g., propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such formulations can be prepared, packaged, or sold in a form suitable for bolus administration or for continuous administration. Injectable formulations can be prepared, packaged, or sold in unit dosage form, such as in ampules, in multi-dose containers containing a preservative, or in single-use devices for auto-injection or injection by a medical practitioner.

Formulations for parenteral administration include suspensions, solutions, emulsions in oily or aqueous vehicles, pastes, and implantable sustained-release or biodegradable formulations. Such formulations can further comprise one or more additional ingredients including suspending, stabilizing, or dispersing agents. In one embodiment of a formulation for parenteral administration, the CXCL12₁ monomer or CXCL12₂ dimer is provided in dry (i.e., powder or granular) form for reconstitution with a suitable vehicle (e.g., sterile pyrogen-free water) prior to parenteral administration of the reconstituted composition.

The pharmaceutical compositions can be prepared, packaged, or sold in the form of a sterile injectable aqueous or oily suspension or solution. This suspension or solution can be formulated according to the known art. Such sterile injectable formulations can be prepared using a non-toxic parenterally-acceptable diluent or solvent, such as water or 1,3-butanediol, for example. Other acceptable diluents and solvents include Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of biodegradable polymer systems. Compositions for sustained release or implantation can comprise pharmaceutically acceptable polymeric or hydrophobic materials such as an emulsion, an ion exchange resin, a sparingly soluble polymer, or a sparingly soluble salt.

The CXCL12₁ monomer or CXCL12₂ dimer of the present invention may also contain adjuvants such as suspending, preserving, wetting, emulsifying, and/or dispersing agents, including, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, such as aluminum monostearate and/or gelatin.

Dosage forms can include solid or injectable implants or depots. In preferred embodiments, the implant comprises an effective amount of the CXCL12₁ monomer and a biodegradable polymer. In preferred embodiments, a suitable biodegradable polymer can be selected from the group consisting of a polyaspartate, polyglutamate, poly(L-lactide), a poly(D,L-lactide), a poly(lactide-co-glycolide), a poly(ε-caprolactone), a polyanhydride, a poly(beta-hydroxy butyrate), a poly(ortho ester) and a polyphosphazene. In other embodiments, the implant comprises an effective amount of the CXCL12₁ monomer or CXCL12₂ dimer and a silastic polymer. The implant provides the release of an effective amount of CXCL12₁ monomer or CXCL12₂ dimer for an extended period ranging from about one week to several years.

Solid dosage forms for oral administration include capsules, tablets, powders, and granules. In such solid dosage forms, the CXCL12₁ monomer or CXCL12₂ dimer is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising the active ingredient can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets can be made by molding, in a suitable device, a mixture of the active ingredient, a pharmaceutically acceptable carrier, and at least sufficient liquid to moisten the mixture.

Tablets may be manufactured with pharmaceutically acceptable excipients such as inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets can be non-coated or coated using known methods to achieve delayed disintegration in the gastrointestinal tract of a human, thereby providing sustained release and absorption of the active ingredient. By way of example, a material such as glyceryl monostearate or glyceryl distearate can be used to coat tablets. Further by way of example, tablets can be coated using methods described in U.S. Pat. Nos. 4,256,108; 4,160,452; and U.S. Pat. No. 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Solid dosage forms such as tablets, dragees, capsules, and granules can be prepared with coatings or shells, such as enteric coatings and others well known in the art. They may also contain opacifying agents, and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Solid compositions of a similar type may also be used as fillers in soft or hard filled gelatin capsules using such excipients as lactose or milk sugar, as well as high molecular weight polyethylene glycols, and the like. Hard capsules comprising the active ingredient can be made using a physiologically degradable composition, such as gelatin. Such hard capsules comprise the active ingredient, and can further comprise additional ingredients including, for example, an inert solid diluent such as calcium carbonate, calcium phosphate, or kaolin. Soft gelatin capsules comprising the active ingredient can be made using a physiologically degradable composition, such as gelatin. Such soft capsules comprise the active ingredient, which can be mixed with water or an oil medium such as peanut oil, liquid paraffin, or olive oil.

Dose Requirements.

In particular embodiments, a preferred range for therapeutically or prophylactically effective amounts of CXCL12₁ or CXCL12₂ may include 0.1 nM-0.1M, 0.1 nM-0.05M, 0.05 nM-15 μM or 0.01 nM-10 μM. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The amount of CXCL12₁ monomer or CXCL12₂ dimer in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such as active compound for the treatment of sensitivity in individuals.

Methods of Use.

The invention also provides corresponding methods of use, including methods of medical treatment, in which a therapeutically effective dose of CXCR12 locked monomer (e.g., CXCL12₁) or CXCL12 locked dimer (e.g., CXCL12₂) is administered in a pharmacologically acceptable formulation. Accordingly, the invention also provides therapeutic compositions comprising the CXCL12₁ or CXCR12₂ and a pharmacologically acceptable excipient or carrier, as described above. The therapeutic composition may advantageously be soluble in an aqueous solution at a physiologically acceptable pH.

In one embodiment, the invention provides a method of treating capillary leak syndrome in a subject comprising administering to the subject a therapeutically effective amount of a composition comprising the CXCL12₁ monomer or CXCL12₂ dimer. By “capillary leak syndrome” we mean escape of the blood plasma through capillary wall, from the blood circulatory system to surrounding tissues, muscle compartments, organs or body cavities. Capillary leak syndrome may be associated with sepsis, autoimmune disease, differentiation syndrome, engraftment syndrome, hemophagocytic lymphohistiocytosis, ovarian hyperstimulation syndrome, viral hemorrhagic fevers, snake bites, and ricin poisoning. Capillary leak syndrome is also associated with impaired endothelial barrier function which leads to leakage of plasma into surrounding tissues. Lung vascular leakage and impairment of lung endothelial permeability is a hallmark in the development of ARDS. In one embodiment, the invention provides a method of treating acute respiratory distress syndrome (ARDS) in a subject comprising administering to the subject a therapeutically effective amount of a composition comprising the CXCL12₁ monomer or CXCL12₂ dimer. By “acute respiratory distress syndrome (ARDS)” we mean the clinical phenotype associated with various pathologies such as trauma, pneumonia, and sepsis characterized by diffuse injury to endothelial cells which form the barrier of the alveoli in the lungs, surfactant dysfunction, activation of the innate immune system response, and dysfunction of the body's regulation of clotting and bleeding.

By “subject” we mean mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, fish and the like. The term “subject” does not denote a particular age or sex.

By “treating” we mean the management and care of a subject for the purpose of combating the disease, condition, or disorder including decreasing, ameliorating or improving of one or more symptom associated with the disease. In the present disclosure, the term “treating” includes reducing, inhibiting or ameliorating capillary leak syndrome or ARDS in a subject in need thereof. For example, treating includes reducing or inhibiting at least one symptom of capillary leak syndrome or ARDS. The terms encompasses palliative treatments. Treating includes the administration of a compound of the present invention to inhibit, reduced, ameliorate and/or improve the onset of the symptoms or complications, alleviating the symptoms or complications, or eliminating the disease, condition, or disorder.

Symptoms of capillary leak syndrome and ARDS are known in the art, and the methods described herein can be used to reduce, inhibit or alleviate at least one symptom of the disease. Symptoms of capillary leak syndrome (SCLS) include, but are not limited to, for example, low blood pressure (hypotension), hypoalbuminemia, decrease in plasma volume (hemoconcentration), fatigue, nausea, abdominal pain, extreme thirst, increase in body weight, elevated white blood count, fluid accumulation in lower limbs, watery stool, among others. Symptoms of ARDS include, but are not limited to, for example, shortness of breath, cough, fever, fast heart rates, rapid breathing, chest pain, decreased oxygen levels, and pathological symptoms, including, for example, severe alveolar congestion, presence of hemorrhage, interstitial edema and increased alveolar wall thickness, among others.

By “prevent” or “preventing” we mean prophylactic or preventive measures intended to inhibit undesirable physiological changes or the development of capillary leak syndrome or ARDS. In exemplary embodiments, preventing capillary leak syndrome or ARDS comprises initiating the administration of a prophylactically effective amount of a composition comprising the CXCL12₁ monomer or CXCL12₂ dimer at a time prior to the appearance or existence of capillary leak syndrome or ARDS such that capillary leak syndrome or ARDS, or their symptoms, pathological features, consequences, or adverse effects do not occur. In such cases, a method of the invention for preventing capillary leak syndrome or ARDS comprises administering a composition comprising the CXCL12₁ monomer or CXCL12₂ dimer to a subject in need thereof prior to exposure of the subject to factors that influence the development of capillary leak syndrome or ARDS.

By “ameliorate”, “amelioration”, “improvement” or the like we mean a detectable improvement or a detectable change consistent with improvement occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range about between any two of these values. Such improvement or change may be observed in treated subjects as compared to subjects not treated with the CXCL12₁ monomer or CXCL12₂ dimer, where the untreated subjects have, or are subject to developing, the same or similar disease, condition, symptom or the like. Amelioration of a disease, condition, symptom or assay parameter may be determined subjectively or objectively, e.g., self-assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or by detection of cell migration within a subject. Amelioration may be transient, prolonged or permanent or it may be variable at relevant times during or after the CXCL12₁ monomer or CXCL12₂ dimer of the present invention is administered to a subject or is used in an assay or other method described herein or a cited reference, e.g., within about 1 hour of the administration or use of the CXCL12₁ monomer or CXCL12₂ dimer of the present invention to about 3, 6, 9 months or more after a subject(s) has received the CXCL12₁ monomer or CXCL12₂ dimer of the present invention.

By “modulation” of, e.g., a symptom, level or biological activity of a molecule, replication of a pathogen, cellular response, cellular activity or the like means that the cell level or activity is detectably increased or decreased. Such increase or decrease may be observed in treated subjects as compared to subjects not treated with the CXCL12₁ monomer or CXCL12₂ dimer of the present invention, where the untreated subjects have, or are subject to developing, the same or similar disease, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or about within any range about between any two of these values. Modulation may be determined subjectively or objectively, e.g., by the subject's self-assessment, by a clinician's assessment or by conducting an appropriate assay or measurement, including, e.g., quality of life assessments or suitable assays for the level or activity of molecules, cells or cell migration within a subject. Modulation may be transient, prolonged or permanent or it may be variable at relevant times during or after the CXCL12₁ monomer or CXCL12₂ dimer of the present invention is administered to a subject or is used in an assay or other method described herein or a cited reference, e.g., within about 1 hour of the administration or use of the CXCL12₁ monomer or CXCL12₂ dimer of the present invention to about 3, 6, 9 months or more after a subject(s) has received the CXCL12₁ monomer of the present invention.

By “administering” we mean any means for introducing the CXCL12₁ monomer or CXCL12₂ dimer of the present invention into the body, preferably into the systemic circulation. Examples include but are not limited to oral, buccal, sublingual, pulmonary, transdermal, transmucosal, as well as subcutaneous, intraperitoneal, intravenous, and intramuscular injection.

By “therapeutically effective amount” we mean an amount effective, at dosages and for periods of time necessary, to achieve the desired therapeutic result, such as reduction or reversal of capillary leak syndrome or acute respiratory distress syndrome. A therapeutically effective amount of CXCL12₁ or CXCL12₂ may vary according to factors such as the disease state, age, sex, and weight of the subject, and the ability of CXCL12₁ or CXCL12₂ to elicit a desired response in the subject. Dosage regimens may be adjusted to provide the optimum therapeutic response. A therapeutically effective amount is also one in which any toxic or detrimental effects of CXCL12₁ or CXCL12₂ are outweighed by the therapeutically beneficial effects.

A “prophylactically effective amount” refers to an amount effective, at dosages and for periods of time necessary, to achieve the desired prophylactic result, such as preventing or inhibiting capillary leak syndrome or ARDS. A prophylactically effective amount can be determined as described above for the therapeutically effective amount. Typically, since a prophylactic dose is used in subjects prior to or at an earlier stage of disease, the prophylactically effective amount will be less than the therapeutically effective amount.

The present invention has been described in terms of one or more preferred embodiments, and it should be appreciated that many equivalents, alternatives, variations, and modifications, aside from those expressly stated, are possible and within the scope of the invention.

### Example 1

The present examples demonstrates that effects if CXCR4/ACKR3 ligands, including monomeric CXCL12₁, on lung endothelial barrier function.

Materials and Methods

Proteins, peptides, and reagents-AMD3100 was purchased from Sigma-Aldrich, CXCL12 and CXCL11 from Protein Foundry, ubiquitin from R&D Systems, TC14012 from Tocris Bioscience and human alpha thrombin from Enzyme Research Laboratories. Recombinant CXCL12 variant proteins were expressed in E. coli, refolded, purified and verified by NMR and high-resolution mass spectrometry as previously described [32].

Cells and cell lines—Human primary pulmonary artery endothelial cells (hPPAEC) (ATCC, PCS-100-022) and the human lung microvascular endothelial cell line HULEC-5a (ATCC, CRL-3244) were cultured in vascular cell basal medium (ATCC, PCS-100-030) with endothelial cell growth kit-VEGF (ATCC, PCS-100-041). The HTLA cell line, a HEK293 cell line stably expressing a tTA-dependent luciferase reporter and a β-arrestin2-TEV fusion gene [33], was generously provided by the laboratory of Dr. Bryan Roth and maintained in high glucose Dulbecco's Modified Eagle's Medium supplemented with 10% (vol/vol) FBS, 1× non-essential amino acids, 100 U/mL penicillin, 100 μg/mL streptomycin, 50 μg/mL hygromycin B, and 2 μg/mL puromycin. All cells were cultured at 37° C., 5% CO2 in a humidified atmosphere.

In vitro vascular permeability assays—Permeability assays were obtained from Millipore (ECM642) and performed as per manufacturer's instructions. In brief, 96-well collagen-coated permeability assay plates were pre-hydrated for 15 min, 5×105 cells were seeded on each well and grown to a confluent monolayer for 48 hours. Fluorescein isothiocyanate (FITC)-dextran (20 μg/mL) was then added on top of the monolayer and the amount of FITC-dextran that permeated through the monolayer was quantified by measuring fluorescence in a Synergy 2 Multi-mode Microplate Reader (BioTek, Winooski, Vt.) at various time points over a 255 min time period.

Proximity ligation assays (PLS)—PLA were performed as described in detail previously [34-36], utilizing mouse anti-ACKR3 (R&D MAB42273) and goat anti-CXCR4 (Abcam Ab1670). The antibodies have been validated for their receptor target previously [35-37]. PLA signals (λ_{excitation/emission} 598/634 nm)) were identified as red spots under a fluorescence microscope.

Presto-Tango β-arrestin 2 recruitment assay—The PRESTO-Tango (parallel receptorome expression and screening via transcriptional out-put, with transcriptional activation following arrestin translocation) assay was performed as recently described [33]. The Tango plasmids were a gift from Dr. Bryan Roth (all from Addgene). HTLA cells (2.5×105/well) were seeded in a 6-well plate and transfected with 1.5 μg of the Tango plasmids using Lipofectamine 3000 (ThermoScientific). The following day, transfected HTLA cells (1×105 cells/well) were plated onto Poly-L-Lysine pre-coated 96-well micro-plates and allowed to attach to the plate surface for at least 4 hours prior to treatment. Proteins used for treatment were prepared in twice the final concentration in culture media, added at a 1:1 vol/vol ratio and incubated overnight at 37° C., 5% CO2 in a humidified environment. The following morning, media was removed from cell culture plates and replaced with a 100 μL 1:5 mixture of Bright-Glo (Promega) and 1×HBSS, 20 mM HEPES solution. Plates were then incubated at room temperature before measuring luminescence on a Biotek Synergy II plate reader.

SDS-polyacrylamide gel electrophoresis (PAGE)—SDS-PAGE was performed utilizing pre-cast mini-PROTEAN TGX gels (Bio-Rad). Lanes were loaded with 1 ug of each protein in 25 μL of Laemmli sample buffer with or without 10% 2-mercaptoethanol (Sigma Aldich) after boiling for 5 min.

Data analysis—Data are expressed as mean±SEM from n independent experiments that were performed on different days. Data were analyzed with unpaired Student's t test, one- or two-way analyses of variance with Bonferroni's multiple comparison post hoc test, as appropriate. Dose-response curves were generated using nonlinear regression analyses. All analyses were performed with the GraphPad-Prism 7 software. A two-tailed P<0.05 was considered significant.

Results

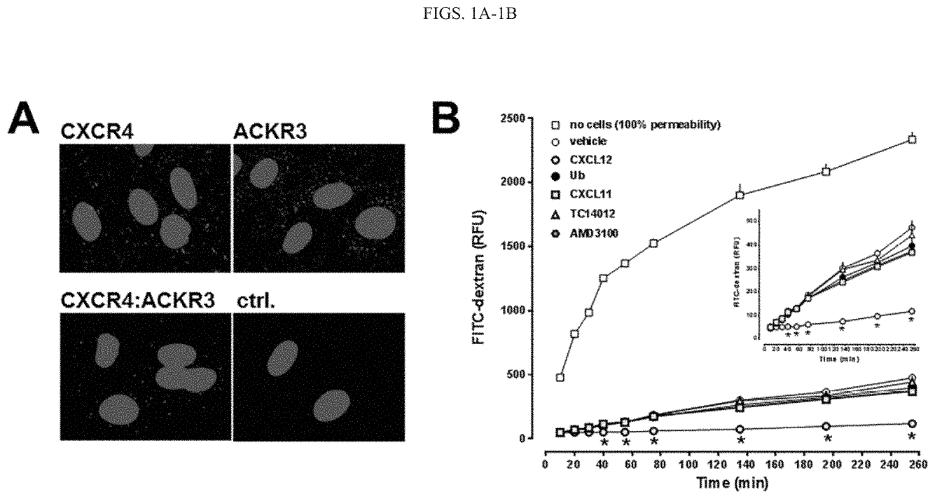

To confirm that CXCR4 and ACKR3 are expressed in hPPAEC and to assess whether both receptors form heteromeric complexes, we performed PLA to detect individual receptors and receptor-receptor interactions at single molecule resolution. As shown in FIG. 1A, we observed positive PLA signals for both receptors individually and for CXCR4:ACKR3 heteromeric complexes. We then tested the effects of a panel of CXCR4 and ACKR3 ligands on hPPAEC mono-layer permeability in transwell-permeability assays with FITC-dextran (FIG. 1B). Ubiquitin, a non-cognate CXCR4 agonist that does not bind to ACKR3, CXCL11, an ACKR3 and CXCR3 agonist, TC14012, a synthetic CXCR4 antagonist and ACKR3 agonist, and AMD3100, a CXCR4 antagonist and allosteric ACKR3 agonist, did not affect hPPAEC permeability [26, 38-41]. In contrast, CXCL12 enhanced hPPAEC barrier function.

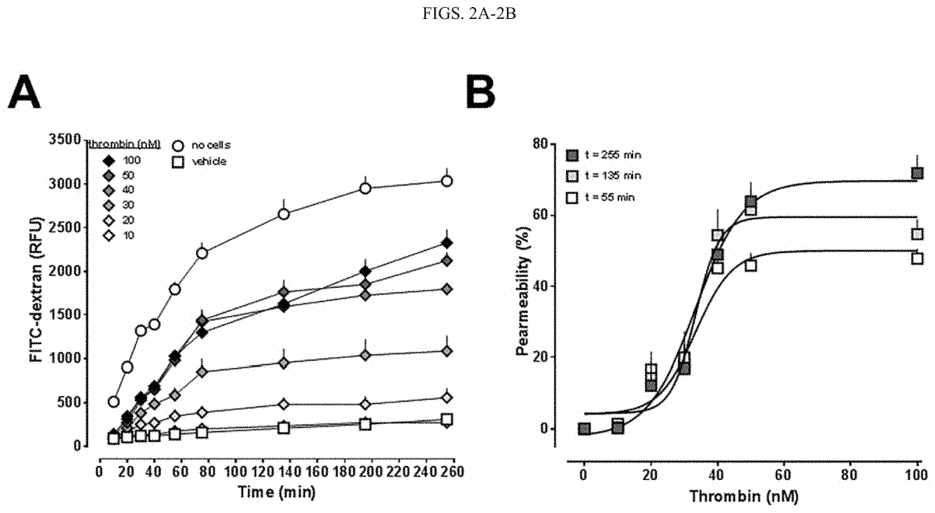

To be able to assess the effects of CXCL12 on thrombin-induced impairment of hPPAEC barrier function under optimized conditions, we determined the dose-response characteristics for thrombin in the permeability assay. The effects of thrombin (10-100 nM) on hPPAEC monolayer permeability are shown in FIG. 2A. Thrombin dose- and time-dependently induced permeability of the hPPAEC monolayer. The time to reach plateau for the permeability-inducing effects of thrombin increased with increasing thrombin concentrations (20 nM-55 min; 30 nM-75 min; 40 nM-135 min; 50 nM and 100 nM→255 min). Based on the results from FIG. 2A, we analyzed the dose-effect relationship for thrombin-induced impairment of lung vascular endothelial cell barrier function at 55 min, 135 min and 255 min (FIG. 2B). The thrombin-mediated effects showed a sigmoidal dose-effect relationship at all time points. The EC50 for thrombin-induced impairment of hPPAEC barrier function was 30±2 nM after 55 min, 33±2 nM after 135 min and 36±2 nM after 255 min. The maximal impairment of endothelial barrier function (100% permeability=measured permeability in the absence of hPPAEC) reached 49±3% at 55 min and 59±4% and 72±4% at 135 min and 255 min, respectively.

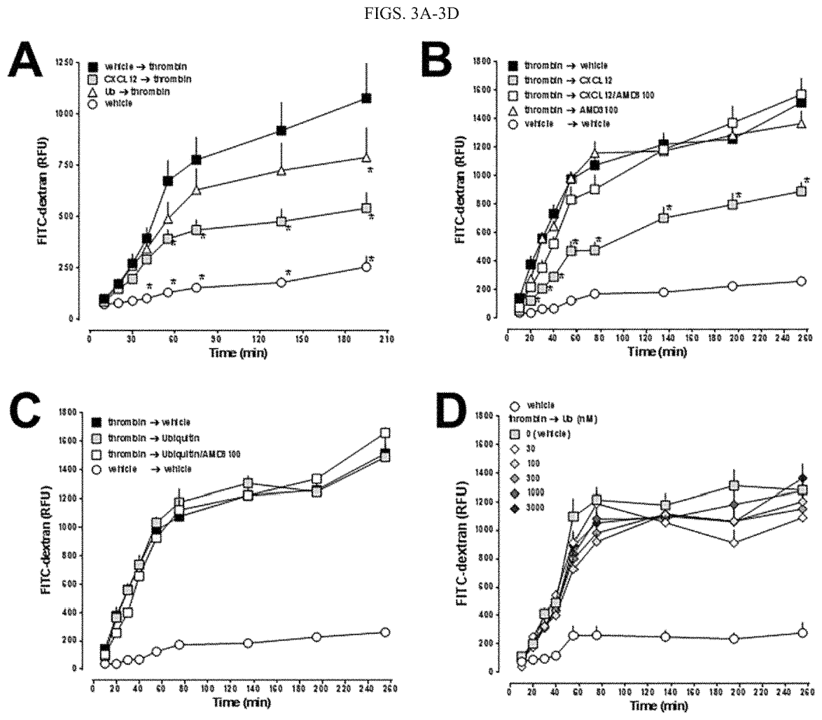

We then tested whether pre-treatment with CXCL12 and ubiquitin influences hyper-permeability of hPPAEC induced by a sub-maximal dose of thrombin. hPPAEC were treated for 10 min with either 100 nM of CXCL12 or ubiquitin, followed by the addition of thrombin (FIG. 3A). Pre-treatment with both CXCR4 agonists significantly attenuated thrombin-induced hyper-permeability of hPPAEC. CXCL12 was more efficacious than ubiquitin in this assay. The effects of CXCL12 and ubiquitin when added after thrombin treatment of hPPAEC are shown in FIGS. 3B and 3C. CXCL12 significantly attenuated thrombin-induced permeability of hPPAEC and this effect could be antagonized with the CXCR4 antagonist AMD3100. AMD3100 treatment alone did not affect thrombin-mediated hyper-permeability of hPPAEC (FIG. 3B). In contrast to CXCL12, ubiquitin-treatment and ubiquitin plus AMD3100-treatment did not modulate thrombin-induced hyper-permeability when tested in parallel experiments (FIG. 3C). To exclude that the dose-effect relationship for ubiquitin is different from the dose-effect relationship for CXCL12, we tested ubiquitin in various concentrations (30 nM-3 μM), including concentrations above the KD-value of ubiquitin for CXCR4 binding [39]. Ubiquitin treatment, however, did not attenuate thrombin-induced hyper-permeability of the hPPAEC monolayer at any tested concentration under these experimental conditions (FIG. 3D).

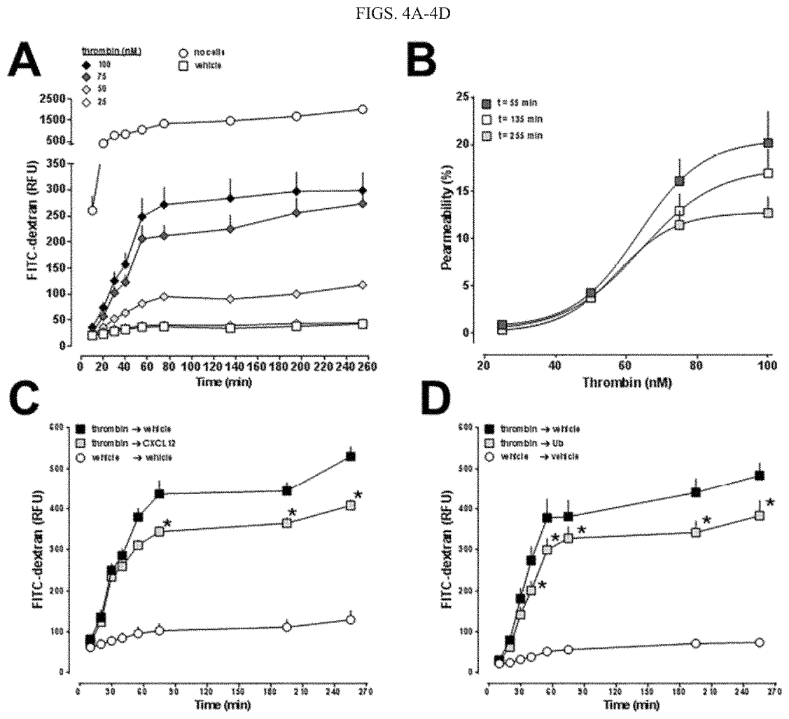

As observed in hPPAEC, thrombin also dose- and time-dependently induced permeability in the human lung microvascular endothelial cell line HULEC-5a (FIGS. 4A and 4B). When com-pared with hPPAEC, potency and efficacy of thrombin to induce permeability were reduced in HULEC-5a. The EC50 for thrombin-induced impairment of HULEC-5a barrier function was 64±7 nM after 55 min, 64±6 nM after 135 min and 57±5 nM after 255 min. Addition of CXCL12 and ubiquitin after treatment of HULEC-5a cells with a sub-maximal dose of thrombin significantly reduced thrombin-mediated impairment of endothelial cell barrier function (FIGS. 4C and 4D). The protective effects of both CXCR4 agonists on thrombin-induced permeability were comparable in HULEC-5a cells; CXCL12, however, was less efficacious in HULEC-5a cells than in hPPAEC.

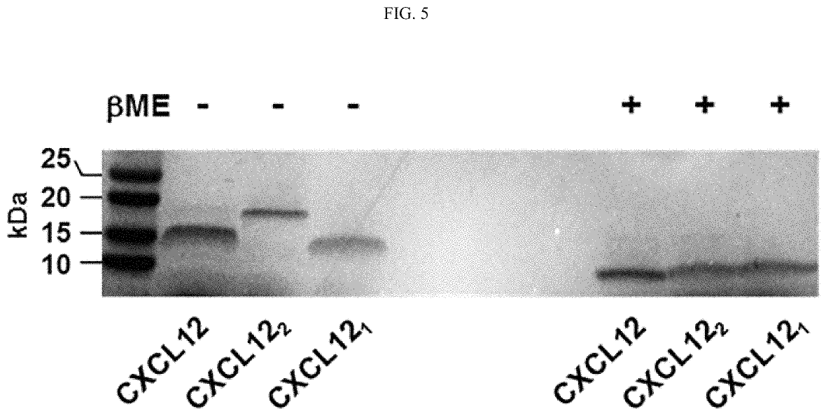

Next, we utilized the Presto-Tango β-arrestin 2 recruitment assay to assess CXCR4 and ACKR3 agonist activities of the natural splice variants CXCL12a and CXCL12β, of truncated CXCL12 (3-68) and the engineered constitutively monomeric (CXCL12₁) and dimeric (CXCL12₂) CXCL12 variants. To confirm the dimeric and monomeric structure of the CXCL12₁ and CXCL12₂ variants, we performed polyacrylamide gel electrophoresis (PAGE) under non-reducing and reducing conditions (FIG. 5). Consistent with the mono- and dimeric nature of the CXCL12 variants [27, 29], the migration position of CXCL12₂ was close to 20 kDa under non-reducing conditions, whereas CXCL12 and CXCL12₁ migrated to a position corresponding to a lower molecular mass. It should be noted that a faint band migrating at the position of CXCL12₂ was visible in non-reducing SDS-PAGE with CXCL12 (loaded at 4 μM), which is consistent with its dimerization Kd of 140 μM [42]. Under reducing conditions all three proteins showed an identical migration position, corresponding to the monomeric molecular mass of approximately 8 kDa.

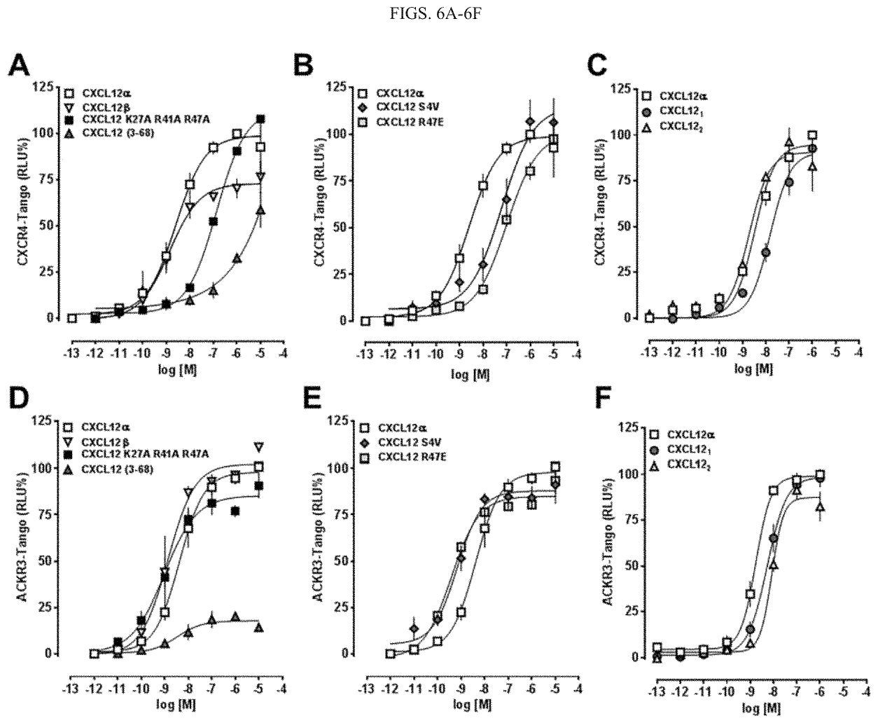

In addition, we tested CXCL12 S-S4V, a protease resistant mutant, CXCL12 K27A/R41A/R47A, which shows significantly reduced heparan sulfate proteoglycan binding properties, and CXCL12 R47E, which activates CXCR4 with reduced potency, as compared with CXCL12α. The dose-response curves are shown in FIG. 6 and Table 1 summarizes the corresponding EC50 concentrations and top plateau values (efficacy) for each protein.

All proteins except CXCL12₃₋₆₈, which lacked relevant CXCR4 activity, showed comparable efficacy to recruit β-arrestin 2 to CXCR4. There were no statistically significant differences between the EC50 concentrations for CXCL12α, CXCL12β, CXCL12₁ and CXCL12₂ in the CXCR4 Presto-Tango assay. The potencies of CXCL12 S-S4V, CXCL12 R47E and CXCL12 K27A/R41A/R47A were significantly lower than the potency of CXCL12α to recruit β-arrestin 2 to CXCR4.

In contrast, all proteins induced β-arrestin 2 recruitment to ACKR3 with an EC50 in the low nM range (p>0.05 for all vs. CXCL12α). While the efficacy for β-arrestin 2 recruitment to ACKR3 was significantly reduced for CXCL12 (3-68), the efficacies of all other proteins for β-arrestin 2 recruitment to ACKR3 were comparable.

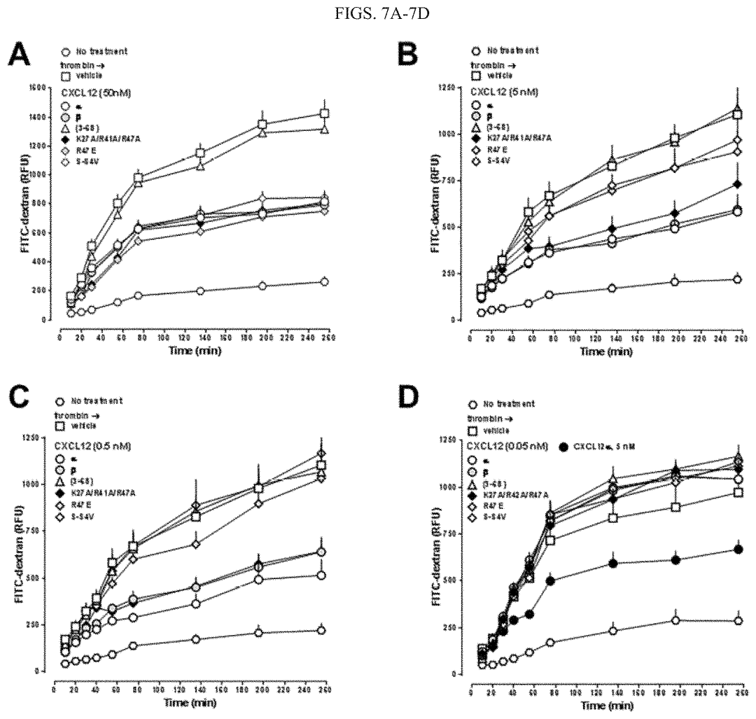

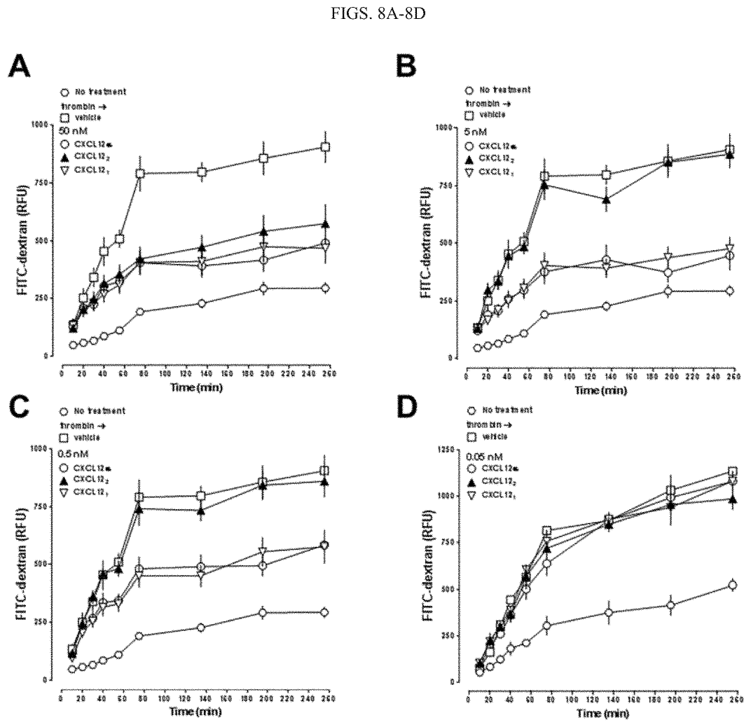

FIGS. 7 and 8 show the effects of the proteins on thrombin-mediated impairment of hPPAEC barrier function when tested in concentrations between 0.05-50 nM in parallel experiments. CXCL12 (3-68) did not attenuate thrombin-induced impairment of hPPAEC barrier function. All other proteins inhibited thrombin-mediated impairment of hPPAEC barrier function with a similar time-dependency of their effects.

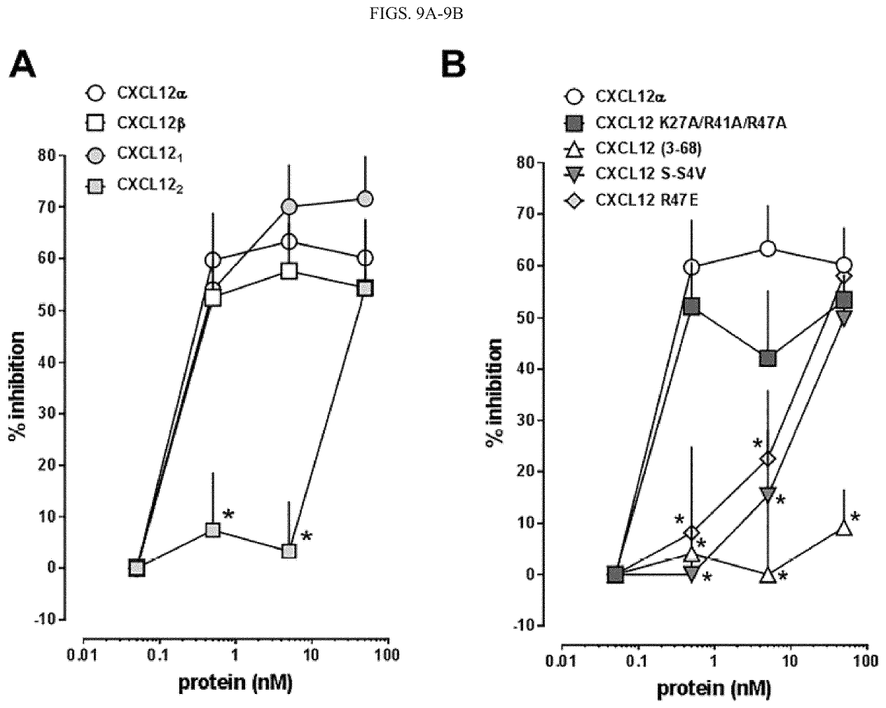

FIG. 9 shows the comparison of their end-point (t=255 min) dose-response profiles. Except for CXCL12 (3-68), the efficacies of all other proteins to inhibit thrombin-induced impairment of hPPAEC barrier function were comparable. CXCL12α, CXCL12β, CXCL12₁ and CXCL12 K27A/R41A/R47A showed similar potencies to inhibit thrombin-mediated hyper-permeability of hPPAEC with EC50 concentrations between 0.05-0.5 nM. The potencies of CXCL12 R47E and CXCL12 S-S4V were one order of magnitude lower (EC50 between 0.5-50 nM). CXCL12₂ affected thrombin-mediated hPPAEC barrier impairment only at a concentration of 50 nM.

Discussion

In the present study, we evaluated the effects of CXCR4 and ACKR3 ligands on the barrier function of human lung endothelial cells. CXCL12 has previously been described to enhance transendothelial electrical resistance, a surrogate marker of endothelial barrier function, of bovine aortic, human pulmonary artery and umbilical vein endothelial cells [22]. Furthermore, pre-treatment of bovine aortic endothelial cells with CXCL12 has been reported to attenuate thrombin-induced FITC-dextran transfer in transwell permeability assays. Likewise, co-treatment of human microvascular endothelial cells with CXCL12 or CTCE-0214, a synthetic CXCL12 analogue, plus thrombin attenuated the reduction of transendothelial resistance that was detectable with thrombin alone [15, 22]. Our observations from the present study are in agreement with previous reports and now provide direct evidence that CXCL12 enhances barrier function of hPPAEC in the absence of permeability-inducing agents. In addition, we demonstrate that pre-treatment of hPPAEC with CXCL12 and with the non-cognate CXCR4 agonist ubiquitin, which does not bind to ACKR3 [38], attenuates thrombin-mediated hPPAEC barrier function impairment. These findings provide a possible mechanism underlying lung protective effects of intravenous CXCL12 pre-treatment in an oleate-induced lung injury model in rabbits and of ubiquitin pre-treatment in an endotoxic shock model in pigs

Although pre- and co-treatment experiments provide information on possible preventive properties of CXCR4 agonists, such experiments are unable to address therapeutic potential. Thus, we performed post-treatment experiments and detected that activation of CXCR4 after thrombin-exposure of hPPAEC and HULEC5a cells attenuates thrombin-mediated impairment of lung endothelial barrier function. These findings support the concept that CXCR4 agonists have therapeutic potential to limit thrombin-mediated pulmonary vascular leakage, which likely contributed to lung protective effects of CXCR4 agonists that have been observed in various models when administered after the insult [15, 17, 18, 21, 43].

In contrast to CXCL12, the non-cognate CXCR4 agonist ubiquitin did not enhance hPPAEC barrier function in the absence of thrombin. As compared with CXCL12, ubiquitin was less efficacious to reduce thrombin-mediated barrier function impairment in pre-treatment experiments with hPPAEC, showed similar efficacy to protect barrier function after thrombin exposure of HUELC5a and failed to protect barrier function after thrombin expo-sure of hPPAEC. These findings could be explained by ubiquitin's lower affinity for and weaker agonist activity at CXCR4, as compared with CXCL12 [38, 39, 44-46].

Recently, we provided evidence that ubiquitin functions as a biased CXCR4 agonist, which does not recruit β-arrestin 2 to CXCR4 [47]. Thus, it appears also possible that the differences between CXCL12 and ubiquitin that we observed in the present study reflect differences in functional outcomes of balanced and biased CXCR4 signaling in lung endothelial cells.

Because none of the ACKR3 agonists affected hPPAEC barrier function and AMD3100 abolished protective effects of CXCL12 on thrombin-mediated barrier function impairment, activation of ACKR3 alone appears not to contribute to the observed effects.

Previously, CXCR4 has been shown to form heteromeric complexes with ACKR3 in expression systems and in human vascular smooth muscle cells [34-36, 48, 49]. Our present finding that PLA signals for CXCR4 and ACKR3 interactions are also detectable in hPPAEC suggests the existence of such endogenous receptor heteromers in the lung endothelium. Thus, another explanation for the observed differences between CXCL12 and ubiquitin could be that simultaneous activation of CXCR4 and ACKR3 within the heteromeric complex is more efficacious to reduce thrombin-mediated endothelial barrier impairment than activation of CXCR4 alone. To address this possibility, detailed mechanistic studies to elucidate the roles of the CXCR4: ACKR3 heteromer will be required in the future. Such experiments, however, are beyond the scope of the present study.

Among the CXCL12 variants that we tested, only CXCL12 (3-68) lacked relevant CXCR4 activity, showed significantly reduced efficacy to activate ACKR3 in Presto-Tango assays and did not attenuate thrombin-induced hPPAEC barrier function impairment. This loss of function is consistent with the loss of function of N-terminal truncated CXCL12 that has been reported previously in other assay systems [23-25].

As expected, both natural CXCL12 splice variants, CXCL12α and CXCL12α, showed comparable properties in Presto-Tango and permeability assays [26]. CXCL12 exists as a monomer at low concentrations and forms dimers at high concentrations or when bound to heparan sulfate on the endothelial surface [31, 50, 51]. Consistent with previous reports, the constitutive monomeric CXCL12 variant (CXCL12₁) showed a behavior similar to wild type proteins in CXCR4/ACKR3 β-arrestin 2 recruitment assays and in permeability assays [27, 28].

Despite activities of the disulfide-locked dimeric CXCL12 variant CXCL12₂ in β-arrestin 2 recruitment assays for CXCR4 and ACKR3 that were comparable with CXCL12α/β, CXCL12₂ showed significantly reduced potency to attenuate thrombin-induced permeability of hPPAEC. The previous finding that CXCL12₂ binds to ACKR3 with very low affinity is not contradictive to our findings in ACKR3 β-arrestin 2 recruitment assays because maximal bio-logical responses of other GPCRs have been observed at ligand occupancies of only a small fraction of receptors [28, 52, 53] and a large receptor reserve is likely in expression systems, such as the Presto-Tango assay. The effects of CXC12₂ in CXCR4 β-arrestin 2 recruitment assays that we observed using the Presto-Tango cell system, however, are conflicting with previous measurements in intermolecular bioluminescence resonance energy transfer (BRET) assays [28]. The Presto-Tango assay utilizes a transcriptional read-out that is measured several hours after the actual signaling event. Thus, it appears possible that few β-arrestin recruitment events upon ligand binding, which may not generate a significant intermolecular BRET signal, can lead to transcription of luciferase in the Presto-Tango system. Furthermore, as compared to previous intermolecular BRET assays in which cells were exposed to CXCL12₂ for 30 min [28], cells were exposed to CXCL12₂ in our Presto-Tango assays for longer time periods, which may contribute to the observed effects. Irrespective of this discrepancy, the low potency of CXCL12₂ to inhibit thrombin-mediated barrier function impairment in the present study in combination with the previously described lack of chemotactic activity of CXCL12₂ [28, 29] demonstrate that this variant does not induce the complete spectrum of biological effects that are mediated via CXCR4 and/or ACKR3 upon activation with the wild type proteins and the constitutively monomeric variant.

In agreement with the low potency of CXCL12 R47E to activate Ca2+ signaling via CXCR4 [29], we observed that this mutant also induces β-arrestin 2 recruitment to CXCR4 and antagonizes thrombin-mediated hyperpermeability of hPPAEC with reduced potency, but retains ACKR3 activity comparable to wild type proteins. Similarly, the protease resistant mutant CXCL12 S-S4V showed reduced CXCR4 activity in Presto-Tango and permeability assays but retained ACKR3 activity. These findings are in agreement with previous effects of this mutant in CXCR4/ACKR3 β-arrestin recruitment and chemotaxis assays [30]. The observations that CXCL12 R47E and CXCL12 S-S4V showed reduced CXCR4 activity but retained ACKR3 activity further supports the assumption that protection from thrombin-mediated hPPAEC barrier impairment is mediated via CXCR4.

CXCL12 is known to bind to heparin oligosaccharides, which promotes dimerization, interferes with CXCL12 binding to CXCR4 and immobilizes CXCL12 on the endothelial surface to establish a concentration gradient required for cell trafficking [31, 54-56]. CXCL12 K27A/R41A/R47A, which binds heparan sulfates with significantly reduced affinity [31], was the only mutant protein that inhibited thrombin-mediated impairment of hPPAEC barrier function with that same potency as wild type proteins and CXCL12₁. This mutant, however, showed the lowest potency to activate CXCR4 in β-arrestin 2 recruitment assays and retained high potency to activate ACKR3. These data suggest that the K27, R41 and R47 mutations reduced the binding affinity for CXCR4 or the efficacy to induce signaling events at lower concentrations. The high potency of this mutant to reduce thrombin-induced barrier function impairment, however, can be explained by its reduced heparan sulfate binding properties, which reduces the proportion of protein that is immobilized on the surface of hPPAEC and thus, is not available for receptor activation [57]. The latter suggests that CXCL12 binding to heparan sulfate on HTLA cells, which were used in Presto-Tango assays, does not significantly affect CXCR4 binding and signaling. This implies that distinct cell surface heparan sulfate proteoglycan expression patterns between various cell types modulate CXCL12-mediated biological functions.

In conclusion, our findings suggest CXCR4 as a possible drug target to attenuate thrombin-mediated impairment of lung endothelial barrier function, demonstrate that stimulation of human lung endothelial cells with cognate and non-cognate CXCR4 agonists results in functional differences and provide initial information on the structure-function relationship for CXCL12-mediated protection from thrombin-induced barrier function impairment in primary human lung endothelial cells. Our findings indicate that the protective effects of CXCL12 are dictated by its CXCR4 agonist activity and by interactions of distinct protein moieties with heparan sulfate proteoglycans on the endothelial cell surface. Interestingly, in disease conditions that are likely associated with thrombin-induced endothelial permeability impairment, such as sepsis or trauma, systemic CXCL12 concentrations have been reported to increase to levels within the range of the EC50 for CXCL12 to attenuate thrombin-induced barrier function impairment in our permeability assays [58-60]. This may suggest that activation of CXCR4 by its endogenous agonists constitutes a protective mechanism to attenuate endothelial barrier function impairment by thrombin in disease conditions and implies that treatment with exogenous CXCR4 agonists augments this protective response. Our findings are expected to facilitate the development of engineered compounds with improved pharmacological properties to attenuate thrombin-induced vascular leakage in the pulmonary circulation, which may have the potential to attenuate development of lung injury and ARDS.

### REFERENCES

1. Blank R, Napolitano L M. Epidemiology of ARDS and ALI. Crit Care Clin. 2011; 27(3):439-58. https://doi.org/10.1016/j.ccc.2011.05.005 PMID: 21742210.
2. Ranieri V M, Rubenfeld G D, Thompson B T, Ferguson N D, Caldwell E, Fan E, et al. Acute respiratory distress syndrome: the Berlin Definition. JAMA. 2012; 307(23):2526-33. Epub 2012/07/17. https://doi. org/10.1001/jama.2012.5669 PMID: 22797452.
3. Gonzales J N, Lucas R, Verin A D. The Acute Respiratory Distress Syndrome: Mechanisms and Perspective Therapeutic Approaches. Austin J Vasc Med. 2015; 2(1). PMID: 26973981; PubMed Central PMCID: PMC4786180.
4. Dushianthan A, Grocott M P, Postle A D, Cusack R. Acute respiratory distress syndrome and acute lung injury. Postgrad Med J. 2011; 87(1031):612-22. https://doi.org/10.1136/pgmj.2011.118398 PMID: 21642654.
5. Ware L B. Pathophysiology of acute lung injury and the acute respiratory distress syndrome. Semin Respir Crit Care Med. 2006; 27(4):337-49. Epub 2006/08/16. https://doi.org/10.1055/s-2006-948288 PMID: 16909368.
6. Johnson E R, Matthay M A. Acute lung injury: epidemiology, pathogenesis, and treatment. J Aerosol Med Pulm Drug Deliv. 2010; 23(4):243-52. https://doi.org/10.1089/jamp.2009.0775 PMID: 20073554; PubMed Central PMCID: PMC3133560.
7. Coughlin S R. Thrombin signalling and protease-activated receptors. Nature. 2000; 407(6801):258-64. https://doi.org/10.1038/35025229 PMID: 11001069.
8. Alberelli M A, De Candia E. Functional role of protease activated receptors in vascular biology. Vascul Pharmacol. 2014; 62(2):72-81. https://doi.org/10.1016/j.vph.2014.06.001 PMID: 24924409.
9. Kawkitinarong K, Linz-McGillem L, Birukov K G, Garcia J G. Differential regulation of human lung epithelial and endothelial barrier function by thrombin. Am J Respir Cell Mol Biol. 2004; 31(5):517-27. https://doi.org/10.1165/rcmb.2003-04320C PMID: 15284075.
10. Mihara K, Ramachandran R, Saifeddine M, Hansen K K, Renaux B, Polley D, et al. Thrombin-Mediated Direct Activation of Proteinase-Activated Receptor-2: Another Target for Thrombin Signaling. Mol Phar-macol. 2016; 89(5):606-14. https://doi.org/10.1124/mol.115.102723 PMID: 26957205.
11. Gur-Cohen S, Kollet O, Graf C, Esmon C T, Ruf W, Lapidot T. Regulation of long-term repopulating hematopoietic stem cells by EPCR/PAR1 signaling. Ann N Y Acad Sci. 2016; 1370(1):65-81. https://doi.org/10.1111/nyas.13013 PMID: 26928241.
12. Arachiche A, Mumaw M M, de la Fuente M, Nieman M T. Protease-activated receptor 1 (PAR1) and PAR4 heterodimers are required for PAR1-enhanced cleavage of PAR4 by alpha-thrombin. J Biol Chem. 2013; 288(45):32553-62. https://doi.org/10.1074/jbc.M113.472373 PMID: 24097976; PubMed Central PMCID: PMC3820888.
13. Coughlin S R. How the protease thrombin talks to cells. Proc Natl Acad Sci USA. 1999; 96(20):11023-7. PMID: 10500117; PubMed Central PMCID: PMC34235.
14. Zhang P, Gruber A, Kasuda S, Kimmelstiel C, O'Callaghan K, Cox D H, et al. Suppression of arterial thrombosis without affecting hemostatic parameters with a cell-penetrating PAR1 pepducin. Circulation. 2012; 126(1):83-91. https://doi.org/10.1161/CIRCULATIONAHA.112.091918 PMID: 22705889; PubMed Central PMCID: PMC3423084.
15. Guo C, Goodwin A J, Buie J N, Cook J A, Halushka P V, Argraves K, et al. A Stromal Cell-derived Factor 1 alpha Analogue Improves Endothelial Cell Function in Lipopolysaccharide-induced Acute Respiratory Distress Syndrome. Mol Med. 2016. https://doi.org/10.2119/molmed.2015.00240 PMID: 27031787; PubMed Central PMCID: PMCPMC5004713 Medicine, or other interests that might be perceived to influence the results and discussion reported in this paper.
16. Garcia-Covarrubias L, Manning E W 3rd, Sorell L T, Pham S M, Majetschak M. Ubiquitin enhances the Th2 cytokine response and attenuates ischemia-reperfusion injury in the lung. Crit Care Med. 2008; 36 (3):979-82. https://doi.org/10.1097/CCM.0B013E318164E417 PMID: 18209671.
17. Majetschak M, Cohn S M, Nelson J A, Burton E H, Obertacke U, Proctor K G. Effects of exogenous ubiquitin in lethal endotoxemia. Surgery. 2004; 135(5):536-43. https://doi.org/10.1016/j.surg.2003.09.006 PMID: 15118591.
18. Earle S A, Proctor K G, Patel M B, Majetschak M. Ubiquitin reduces fluid shifts after traumatic brain injury. Surgery. 2005; 138(3):431-8. https://doi.org/10.1016/j.surg.2005.06.026 PMID: 16213895.
19. Baker T A, Romero J, Bach H Ht, Strom J A, Gamelli R L, Majetschak M. Effects of exogenous ubiquitin in a polytrauma model with blunt chest trauma. Crit Care Med. 2012. Epub 2012/05/25. https://doi.org/10. 1097/CCM.0b013e3182514ed9 PMID: 22622399.
20. Guo W, Li Z, Xie X, Tan T, Wang S, Xie N, et al. Stromal cell-derived factor-lalpha attenuates oleate-induced acute lung injury in rabbits. Biochem Biophys Res Commun. 2014. https://doi.org/10.1016/j. bbrc.2014.07.033 PMID: 25019980.
21. Nassoiy S P, Babu F S, LaPorte H M, Majetschak M. Pharmacological modulation of C-X-C motif chemokine receptor 4 influences development of acute respiratory distress syndrome after lung ischemia-reperfusion injury. Clin Exp Pharmacol Physiol. 2017. https://doi.org/10.1111/1440-1681.12845 PMID: 28815665.
22. Kobayashi K, Sato K, Kida T, Omori K, Hori M, Ozaki H, et al. Stromal cell-derived factor-1alpha/C-X-C chemokine receptor type 4 axis promotes endothelial cell barrier integrity via phosphoinositide 3-kinase and Rac1 activation. Arterioscler Thromb Vasc Biol. 2014; 34(8):1716-22. https://doi.org/10.1161/ATVBAHA.114.303890 PMID: 24925969.
23. Crump M P, Gong J H, Loetscher P, Rajarathnam K, Amara A, Arenzana-Seisdedos F, et al. Solution structure and basis for functional activity of stromal cell-derived factor-1; dissociation of CXCR4 activation from binding and inhibition of HIV-1. Embo J. 1997; 16(23):6996-7007. https://doi.org/10.1093/emboj/16.23.6996 PMID: 9384579.
24. Janssens R, Mortier A, Boff D, Ruytinx P, Gouwy M, Vantilt B, et al. Truncation of CXCL12 by CD26 reduces its CXC chemokine receptor 4- and atypical chemokine receptor 3-dependent activity on endothelial cells and lymphocytes. Biochem Pharmacol. 2017; 132:92-101. https://doi.org/10.1016/j.bcp. 2017.03.009 PMID: 28322746.
25. Proost P, Struyf S, Schols D, Durinx C, Wuyts A, Lenaerts J P, et al. Processing by CD26/dipeptidyl-peptidase IV reduces the chemotactic and anti-HIV-1 activity of stromal-cell-derived factor-lalpha. FEBS Lett. 1998; 432(1-2):73-6. PMID: 9710254.
26. Bachelerie F, Ben-Baruch A, Burkhardt A M, Combadiere C, Farber J M, Graham G J, et al. International Union of Pharmacology. LXXXIX. Update on the extended family of chemokine receptors and introducing a new nomenclature for atypical chemokine receptors. Pharmacol Rev. 2014; 66(1):1-79. Epub 2013/11/13. https://doi.org/10.1124/pr.113.007724 PMID: 24218476; PubMed Central PMCID: PMC3880466.
27. Ziarek J J, Kleist A B, London N, Raveh B, Montpas N, Bonneterre J, et al. Structural basis for chemokine recognition by a G protein-coupled receptor and implications for receptor activation. Sci Signal. 2017; 10(471). https://doi.org/10.1126/scisignal.aah5756 PMID: 28325822.
28. Drury L J, Ziarek J J, Gravel S, Veldkamp C T, Takekoshi T, Hwang S T, et al. Monomeric and dimeric CXCL12 inhibit metastasis through distinct CXCR4 interactions and signaling pathways. Proc Natl Acad Sci USA. 2011; 108(43):17655-60. Epub 2011/10/13. https://doi.org/10.1073/pnas.1101133108 PMID: 21990345; PubMed Central PMCID: PMC3203819.
29. Veldkamp C T, Seibert C, Peterson F C, De la Cruz N B, Haugner J C 3rd, Basnet H, et al. Structural basis of CXCR4 sulfotyrosine recognition by the chemokine SDF-1/CXCL12. Sci Signal. 2008; 1(37): ra4. https://doi.org/10.1126/scisignal.1160755 PMID: 18799424.
30. Segers V F, Tokunou T, Higgins L J, MacGillivray C, Gannon J, Lee R T. Local delivery of protease-resistant stromal cell derived factor-1 for stem cell recruitment after myocardial infarction. Circulation. 2007; 116(15):1683-92. https://doi.org/10.1161/CIRCULATIONAHA.107.718718 PMID: 17875967.
31. Ziarek J J, Veldkamp C T, Zhang F, Murray N J, Kartz G A, Liang X, et al. Heparin oligosaccharides inhibit chemokine (CXC motif) ligand 12 (CXCL12) cardioprotection by binding orthogonal to the dimerization interface, promoting oligomerization, and competing with the chemokine (CXC motif) receptor 4 (CXCR4) N terminus. J Biol Chem. 2013; 288(1):737-46. https://doi.org/10.1074/jbc.M112.394064 PMID: 23148226; PubMed Central PMCID: PMCPMC3537072.
32. Veldkamp C T, Koplinski C A, Jensen D R, Peterson F C, Smits K M, Smith B L, et al. Production of Recombinant Chemokines and Validation of Refolding. Methods Enzymol. 2016; 570:539-65. https://doi.org/10.1016/bs.mie.2015.09.031 PMID: 26921961; PubMed Central PMCID: PMCPMC4811038.
33. Kroeze W K, Sassano M F, Huang X P, Lansu K, McCorvy J D, Giguere P M, et al. PRESTO-Tango as an open-source resource for interrogation of the druggable human GPCRome. Nat Struct Mol Biol. 2015; 22(5):362-9. https://doi.org/10.1038/nsmb.3014 PMID: 25895059; PubMed Central PMCID: PMCPMC4424118.
34. Evans A E, Tripathi A, Laporte H M, Brueggemann L I, Singh A K, Albee L J, et al. New insights into mechanisms and functions of chemokine (C-X-C motif) receptor 4 heteromerization in vascular smooth mus-cle. Int J Mol Sci. 2016 Jun. 20; 17(5). pii: E971. https://doi.org/10.3390/ijms17060971 PMID: 27331810; PubMed Central PMCID: PMC4926503.
35. Tripathi A, Vana P G, Chavan T S, Brueggemann L I, Byron K L, Tarasova N I, et al. Heteromerization of chemokine (C-X-C motif) receptor 4 with alphalA/B-adrenergic receptors controls alphal-adrenergic receptor function. Proc Natl Acad Sci USA. 2015; 112(13):E1659-68. https://doi.org/10.1073/pnas. 1417564112 PMID: 25775528; PubMed Central PMCID: PMC43 863 52.
36. Albee L J, Eby J M, Tripathi A, Laporte H M, Gao X, Volkman B F, et al. al-Adrenoceptors function within hetero-oligomeric complexes with chemokine receptors ACKR3 and CXCR4 in vascular smooth muscle cells. J Am Heart Assoc. 2017 Aug. 17; 6(8). pii: e006575. https://doi.org/10.1161/JAHA.117.006575 PMID: 28862946; PubMed Central PMCID: PMC5586474.
37. Tripathi A, Gaponenko V, Majetschak M. Commercially available antibodies directed against alpha-adrenergic receptor subtypes and other G protein-coupled receptors with acceptable selectivity in flow cytometry experiments. Naunyn Schmiedebergs Arch Pharmacol. 2015. https://doi.org/10.1007/s00210-015-1196-0 PMID: 26660071; PubMed Central PMCID: PMC4718873.
38. Saini V, Staren D M, Ziarek J J, Nashaat Z N, Campbell E M, Volkman B F, et al. The CXC chemokine receptor 4 ligands ubiquitin and stromal cell-derived factor-lalpha function through distinct receptor interactions. J Biol Chem. 2011; 286(38):33466-77. https://doi.org/10.1074/jbc.M111.233742 PMID: 21757744; PubMed Central PMCID: PMC3190899.
39. Saini V, Marchese A, Majetschak M. CXC chemokine receptor 4 is a cell surface receptor for extracellular ubiquitin. J Biol Chem. 2010; 285(20):15566-76. https://doi.org/10.1074/jbc.M110.103408 PMID: 20228059; PubMed Central PMCID: PMC2865327.
40. Gravel S, Malouf C, Boulais P E, Berchiche Y A, Oishi S, Fujii N, et al. The peptidomimetic CXCR4 antagonist TC14012 recruits beta-arrestin to CXCR7: roles of receptor domains. J Biol Chem. 2010; 285(49):37939-43. Epub 2010/10/20. https://doi.org/10.1074/jbc.C110.147470 PMID: 20956518; PubMed Central PMCID: PMC2992227.
41. Kalatskaya I, Berchiche Y A, Gravel S, Limberg B J, Rosenbaum J S, Heveker N. AMD3100 is a CXCR7 ligand with allosteric agonist properties. Mol Pharmacol. 2009; 75(5):1240-7. Epub 2009/03/04. https://doi.org/10.1124/mo1.108.053389 PMID: 19255243.
42. Ziarek J J, Getschman A E, Butler S J, Taleski D, Stephens B, Kufareva I, et al. Sulfopeptide probes of the CXCR4/CXCL12 interface reveal oligomer-specific contacts and chemokine allostery. ACS Chem Biol. 2013; 8(9):1955-63. https://doi.org/10.1021/cb400274z PMID: 23802178; PubMed Central PMCID: PMCPMC3783652.
43. Baker T A, Romero J, Bach HHt, Strom J A, Gamelli R L, Majetschak M. Effects of exogenous ubiquitin in a polytrauma model with blunt chest trauma. Crit Care Med. 2012; 40(8):2376-84. https://doi.org/10. 1097/CCM.0b013e3182514ed9 PMID: 22622399; PubMed Central PMCID: PMC3886622.
44. Saini V, Marchese A, Tang W J, Majetschak M. Structural determinants of ubiquitin-CXC chemokine receptor 4 interaction. J Biol Chem. 2011; 286(51):44145-52. https://doi.org/10.1074/jbc.M111.298505 PMID: 22039044; PubMed Central PMCID: PMC3243501.
45. Saini V, Romero J, Marchese A, Majetschak M. Ubiquitin receptor binding and signaling in primary human leukocytes. Commun Integr Biol. 2010; 3(6):608-10. https://doi.org/10.4161/cib.3.6.13375 PMID: 21331255; PubMed Central PMCID: PMC3 03 8079.
46. Tripathi A, Saini V, Marchese A, Volkman B F, Tang W J, Majetschak M. Modulation of the CXC chemokine receptor 4 agonist activity of ubiquitin through C-terminal protein modification. Biochemistry. 2013; 52(24):4184-92. https://doi.org/10.1021/bi400254fPMID: 23697661; PubMed Central PMCID: PMC4113718.
47. Eby J M, Abdelkarim H, Albee L J, Tripathi A, Gao X, Volkman B F, et al. Functional and structural consequences of chemokine (C-X-C motif) receptor 4 activation with cognate and non-cognate agonists. Mol Cell Biochem. 2017:1-9. https://doi.org/10.1007/s11010-017-3044-7 PMID: 28455789.
48. Levoye A, Balabanian K, Baleux F, Bachelerie F, Lagane B. CXCR7 heterodimerizes with CXCR4 and regulates CXCL12-mediated G protein signaling. Blood. 2009; 113(24):6085-93. Epub 2009/04/22. https://doi.org/10.1182/blood-2008-12-196618 PMID: 19380869.
49. Decaillot F M, Kazmi M A, Lin Y, Ray-Saha S, Sakmar T P, Sachdev P. CXCR7/CXCR4 heterodimer con-stitutively recruits beta-arrestin to enhance cell migration. J Biol Chem. 2011; 286(37):32188-97. Epub 2011/07/07. https://doi.org/10.1074/jbc.M111.277038 PMID: 21730065; PubMed Central PMCID: PMC3173186.
50. Veldkamp C T, Peterson F C, Pelzek A J, Volkman B F. The monomer-dimer equilibrium of stromal cell-derived factor-1 (CXCL 12) is altered by pH, phosphate, sulfate, and heparin. Protein Sci. 2005; 14
   (4):1071-81. Epub 2005/03/03. https://doi.org/10.1110/ps.041219505 PMID: 15741341; PubMed Central PMCID: PMC2253449.
51. Murphy J W, Cho Y, Sachpatzidis A, Fan C, Hodsdon M E, Lolis E. Structural and functional basis of CXCL12 (stromal cell-derived factor-1 alpha) binding to heparin. J Biol Chem. 2007; 282(13):10018-27. https://doi.org/10.1074/jbc.M608796200 PMID: 17264079; PubMed Central PMCID: PMCPMC3684283.
52. Gifford A N, Bruneus M, Gatley S J, Lan R, Makriyannis A, Volkow N D. Large receptor reserve for cannabinoid actions in the central nervous system. J Pharmacol Exp Ther. 1999; 288(2):478-83. Epub 1999/01/26. PMID: 9918548.
53. Ethier M F, Schaefer O P, Samant N, Yamaguchi H, Madison J M. Muscarinic receptor reserve for inhibition of cAMP accumulation in bovine trachealis cells. Am J Physiol. 1996; 270(2 Pt 1):L199-207. Epub 1996/02/01. PMID: 8779988.
54. Handel T M, Johnson Z, Crown S E, Lau E K, Proudfoot A E. Regulation of protein function by glycosaminoglycans—as exemplified by chemokines. Annu Rev Biochem. 2005; 74:385-410. https://doi.org/10.1146/annurev.biochem.72.121801.161747 PMID: 15952892.
55. Johnson Z, Proudfoot A E, Handel T M. Interaction of chemokines and glycosaminoglycans: a new twist in the regulation of chemokine function with opportunities for therapeutic intervention. Cytokine Growth Factor Rev. 2005; 16(6):625-36. https://doi.org/10.1016/j.cytogfr.2005.04.006 PMID: 15990353.
56. Sadir R, Baleux F, Grosdidier A, Imberty A, Lortat-Jacob H. Characterization of the stromal cell-derived factor-lalpha-heparin complex. J Biol Chem. 2001; 276(11):8288-96. https://doi.org/10.1074/jbc. M008110200 PMID: 11087743.
57. Verkaar F, van Offenbeek J, van der Lee MMC, van Lith L, Watts A O, Rops A, et al. Chemokine cooperativity is caused by competitive glycosaminoglycan binding. J Immunol. 2014; 192(8):3908-14. https://doi.org/10.4049/jimmuno1.1302159 PMID: 24639348; PubMed Central PMCID: PMCPMC4198333.
58. Majetschak M. Extracellular ubiquitin: immune modulator and endogenous opponent of damage-associated molecular pattern molecules. J Leukoc Biol. 2011; 89(2):205-19. https://doi.org/10.1189/jlb. 0510316 PMID: 20689098.
59. Franchini S, Marciano T, Sorlini C, Campochiaro C, Tresoldi M, Sabbadini M G, et al. Serum CXCL12 levels on hospital admission predict mortality in patients with severe sepsis/septic shock. Am J Emerg Med. 2015; 33(12):1802-4. https://doi.org/10.1016/j.ajem.2015.08.047 PMID: 26387470.
60. Chen T J, Wu W Q, Ying G R, Fu Q Y, Xiong K. Serum CXCL12 concentration in patients with severe traumatic brain injury are associated with mortality. Clin Chim Acta. 2016; 454:6-9. https://doi.org/10.1016/j.cca.2015.12.033 PMID: 26724366.

### Example 2

Therapeutic efficacy of the CXCL12-locked monomer (CXCL12₁) and CXCL12-locked dimer (CXCL12₂) was tested for efficacy in ARDS.

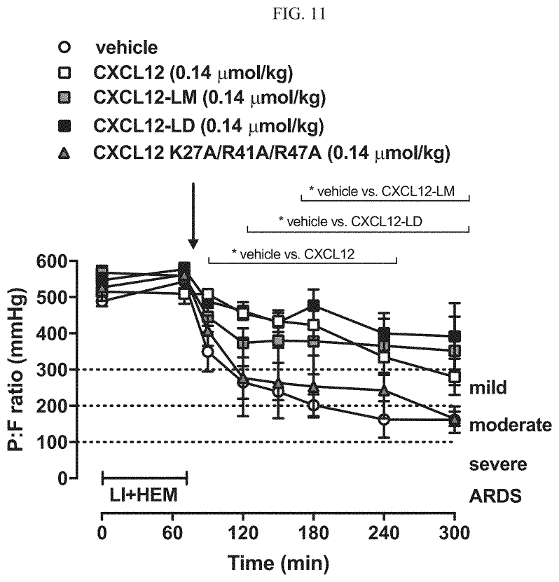

We tested therapeutic efficacy of wild-type CXCL12, CXXL12-locked dimer (LD), CXCL12-locked monomer (LM) and the triple mutant CXCL12 K27A/R41A/R47A in an animal model of acute respiratory distress syndrome (ARDS) (FIG. 11). All drugs were injected in a dose of 0.14 μmol/kg. Male Sprague-Dawley rats were anesthetized, orotracheally intubated, instrumented with arterial/venous catheters, and mechanically ventilated (PEEP 2 mmHg, FiO₂ 1.0). After a right lateral thoracotomy, a suture was tied around the hilum of the right lung. Animals underwent 70 min of right lung ischemia plus hemorrhage to a mean arterial blood pressure (MAP) of 40 mmHg for the last 30 min of lung ischemia. At t=70 min, the suture was removed, animals were ventilated with FiO₂ 1.0, PEEP 5 mmHg and resuscitated with crystalloids to a MAP of 60 mmHg until t=300 min. Vehicle and drugs were injected i.v. within 10 min after reperfusion.

Animals treated with vehicle developed ARDS within 120 min (P:F ratio<300 mmHg) and reached moderate ARDS (P:F ratio<200 mmHg) at the end of the experiment (t=300 min). With CXCL12, development of ARDS was delayed and attenuated. Animals treated with CXCL12 fulfilled criteria for mild ARDS only at the end of the experiment (t=300 min). As compared with vehicle treated animals, P:F ratios were significantly higher with CXCL12 treatment from t=90-240 min. Animals treated with the triple mutant CXCL12 K27A/R41A/R47A were indistinguishable from vehicle treated animals. In contrast, CXCL12-LM and CXCL12-LD treatment prevented development of ARDS. These data suggest CXCL12-LM and CXCL12-LD as promising drug candidates.

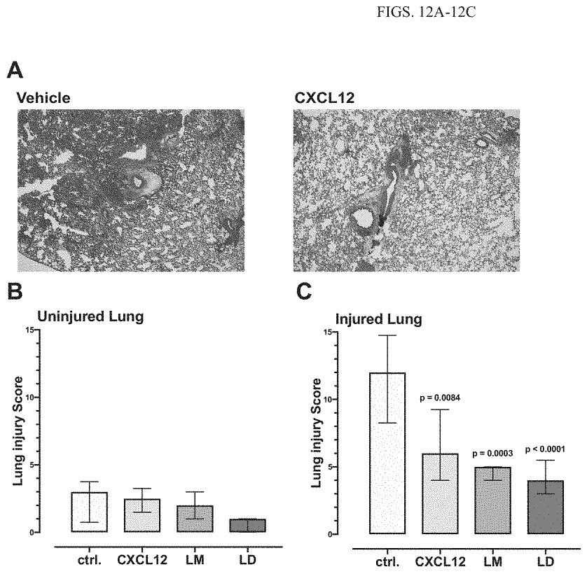

We further evaluated histomorphology of the lungs from animals treated with vehicle, wild-type CXCL12, CXCL12-LD and CXCXL12-LM utilizing a standardized lung injury score. Lung slides stained with hematoxylin and eosin were examined under a light microscope by 4 investigators blinded to the identity of the specimens. The lung injury score was assessed as previously described/. In brief each investigator rendered a score of 0 (no damage) to 4+(maximal damage) based on an injury grading scale composed of six criteria. The slides were assessed for the extent of alveolar congestion, hemorrhage and edema, as well as alveolar and vessel wall PMN infiltration and alveolar wall thickness. FIG. 2A shows representative images from lung slides (injured lungs) of animals treated with vehicle and wild-type CXCL12. Lung injury scores of the uninjured and injured lungs from animals treated with vehicle, wild-type CXCL12, CXCL12-LD and CXCXL12-LM are shown in FIGS. 12A and 12B, respectively.

There were no differences in the histomorphology of the uninjured (left) lungs among groups (FIG. 12B). With vehicle treatment, histology of the injured lung showed severe alveolar congestion, presence of haemorrhage, interstitial oedema and increased alveolar wall thickness (FIGS. 12A and C). These histological signs of lung injury were significantly reduced after treatment with CXCL12, CXCL12-LD and CXCL12-LM.

### References for Example 2

[1] Manning, E. W., 3rd, Patel, M. B., Garcia-Covarrubias, L., Rahnemai-Azar, A. A., Pham, S. M., and Majetschak, M. (2009) Proteasome peptidase activities parallel histomorphological and functional consequences of ischemia-reperfusion injury in the lung, Exp Lung Res 35, 284-295.

We claim:

1. A method of treating capillary leakage syndrome in a subject in need thereof comprising administering to the subject a therapeutically effective amount of a composition comprising a CXCL12α locked dimer polypeptide comprising two monomers locked together by covalent bond to treat capillary leakage syndrome.

2. The method of claim 1, wherein the composition comprises a constitutively monomeric CXCL12 peptide comprising the amino acid sequence of SEQ ID NO:1 wherein the amino acids at positions 55 and 58 are substituted with cysteine to treat capillary leakage syndrome.

3. The method of claim 2, wherein the peptide is CXCL12₁ having the amino acid sequence of SEQ ID NO:2.

4. The method of claim 2, wherein the composition additionally comprises a pharmaceutically acceptable carrier or diluent.

5. The method of claim 2, wherein the subject is a human subject.

6. The method of claim 2, wherein the subject suffers from a disease selected from the group consisting of sepsis, autoimmune disease, differentiation syndrome, engraftment syndrome, hemophagocytic lymphohistiocytosis, ovarian hyperstimulation syndrome, viral hemorrhagic fevers, snake bites, and ricin poisoning which is associated with capillary leakage syndrome.

7. The method of claim 1, wherein the CXCL12α locked dimer polypeptide comprises at least one monomer with a mutation in the amino acid L36, A65, or both 136 and A65 in SEQ ID NO:1 to a cysteine.

8. The method of claim 1, wherein the CXCL12α locked dimer polypeptide comprises monomers locked together at residues L36 and A65 of SEQ ID NO:1.

9. The method of claim 1, wherein the CXCL12α locked dimer polypeptide is SEQ ID NO:3.

10. The method of claim 1, wherein the composition additionally comprises a pharmaceutically acceptable carrier or diluent.

11. The method of claim 1, wherein the subject suffers from a disease selected from the group consisting of sepsis, autoimmune disease, differentiation syndrome, engraftment syndrome, hemophagocytic lymphohistiocytosis, ovarian hyperstimulation syndrome, viral hemorrhagic fevers, snake bites, and ricin poisoning which is associated with capillary leakage syndrome.

12. A method of treating acute respiratory distress syndrome (ARDS) in a subject in need thereof comprising administering to the subject a therapeutically effective amount of a composition comprising a constitutively monomeric CXCL12 peptide comprising the amino acid sequence of SEQ ID NO:1 wherein the amino acids at positions 55 and 58 are substituted with cysteine to treat the ARDS.

13. The method of claim 12, wherein the peptide is CXCL12₁ having the amino acid sequence of SEQ ID NO:2.

14. The method of claim 12, wherein the composition additionally comprises a pharmaceutically acceptable carrier or diluent.

15. The method of claim 12, wherein the subject is a human.

16. A method of treating acute respiratory distress syndrome (ARDS) in a subject in need thereof comprising administering to the subject a therapeutically effective amount of a composition comprising a CXCL12α locked dimer polypeptide comprising two monomers locked together by covalent bond to treat the ARDS.

17. The method of claim 16, wherein the CXCL12α locked dimer polypeptide comprises at least one monomer with a mutation in the amino acid L36, A65, or both 136 and A65 in SEQ ID NO:1 to a cysteine.

18. The method of claim 16, wherein the CXCL12α locked dimer polypeptide comprises monomers locked together at residues L36 and A65 of SEQ ID NO:1.

19. The method of claim 16, wherein the CXCL12α locked dimer polypeptide is SEQ ID NO:3.

20. The method of claim 17, wherein the composition additionally comprises a pharmaceutically acceptable carrier or diluent.


SEQUENCE LISTING (3 sequences)

SEQ ID NO: 1 - protein, 68 aa, Homo sapiens
KPVSLSYRCPCRFFESHVARANVKHLKILNTPNCALQIVARLKNNNRQVCIDPKLKWIQEYLEKALNK

SEQ ID NO: 2 - protein, 68 aa, Artificial Sequence
  CXCL121, a constitutively monomeric CXCL12 variant with L55C and I58C substitutions
KPVSLSYRCPCRFFESHVARANVKHLKILNTPNCALQIVARLKNNNRQVCIDPKCKWCQEYLEKALNK

SEQ ID NO: 3 - protein, 68 aa, Artificial Sequence
  CXCL122, a locked dimer variant of CXCL12 with LL36C and A65C substitutions
KPVSLSYRCPCRFFESHVARANVKHLKILNTPNCACQIVARLKNNNRQVCIDPKLKWIQEYLEKCLNK